(19) United States
(12) Patent Application Publication
(10) Pub. No.: US 2014/0286966 A1
(43) Pub. Date: Sep. 25, 2014

(54) METHODS AND COMPOSITIONS FOR THE TREATMENT OF METABOLIC SYNDROME, OBSTRUCTIVE RESPIRATORY DISORDERS, CANCER AND RELATED DISEASES

(71) Applicant: Youngman Oh, Glen Allen, VA (US)

(72) Inventor: Youngman Oh, Glen Allen, VA (US)

(21) Appl. No.: 14/123,493

(22) PCT Filed: Jul. 6, 2011
(86) PCT No.: PCT/US11/43061

§ 371 Date: Mar. 20, 2014

Related U.S. Application Data
(60) Provisional application No. 61/492,631, filed on Jun. 2, 2011.
(60) Provisional application No. 61/361,577, filed on Jul. 6, 2010.

(51) Int. Cl. A61K 39/395; A61K 35/76; A61K 35/28; A61K 38/17

(52) U.S. Cl. CPC A61K 39/3955; A61K 38/1754; A61K 35/761; A61K 35/28
USPC 424/172.1; 514/8.7; 514/44 R; 424/93.6; 424/93.7; 435/375; 435/455

(57) ABSTRACT

Compositions comprising IGFBP-3 receptor agonists and methods for the treatment of metabolic syndrome, obstructive respiratory disorders, obstructive or inflammatory respiratory disease, cancers and related diseases with IGFBP-3 receptor agonists are presented. A method for interfering with the activity of nuclear factor-kappaB (NF-KB) in a cell, comprising: providing to a cell an effective amount of a composition comprising an IGFBP-3 receptor agonist; and interfering with the activity of NF-KB in the cell is included.

### TECHNICAL FIELD

The invention relates to methods and compositions for the treatment of metabolic syndrome, obstructive respiratory disorders, cancers and related diseases. In particular, the invention relates to compositions comprising IGFBP-3 receptor agonists and methods for the treatment of metabolic syndrome, obstructive respiratory disorders, cancers and related diseases with IGFBP-3 receptor agonists.

### BACKGROUND OF THE INVENTION

Nuclear factor-kappaB (NFκB) signalling, and in particular the dysregulation of NFκB signalling has been implicated in a variety of human disorders. For example, dysregulation of NFκB is thought to play a role in inappropriate immune responses such as autoimmunity and inflammatory diseases. In the light of the proposed role of NFκB dysregulation in certain conditions, it is possible that manipulation of the NFκB signaling pathway may provide a means by which these conditions, including autoimmunity or inflammatory conditions may be treated. One family of such conditions is the family of obstructive respiratory disorders, such as asthma.

Poor nutrition and obesity have become an increasing public health concern as they may affect many metabolic disorders, including heart disease, diabetes, digestive system disorders, and renal disease. Recent findings indicate extensive inter-dependence between metabolic dysregulation, atherosclerosis, inflammation, and innate immunity. These studies show that the causative relationships between obesity, insulin resistance, and atherosclerosis are mediated not only by associated hyperlipidemias but also by co-existing inflammatory states. A central player in these processes is the adipocyte. An intricate link has been demonstrated between metabolic control, innate immunity, and inflammation at the cellular level of the adipocyte. Dysregulation at the cellular level of any one of these transcriptional programs (i.e., metabolic control, innate immunity, and inflammation) can have a profound impact on the other cellular processes.

Obesity is associated with increasing numbers of infiltrating macrophages in adipose tissue (Soukas 2000, Weisburg 2003, and Xu 2003). These adipose tissue macrophages are currently considered to be a major cause of obesity-associated chronic low grade inflammation (Wellen 2003 and 2005) via secretion of a wide variety of inflammatory molecules (Kershaw 2004), including TNF-α (Hotamisligil 1993), IL-6 (Fernandez-Real 2003), monocyte chemoattractant protein-1 (MCP-1) (Takahashi 2003 and Christiansen 2005), and plasminogen activator inhibitor-1 (Shimomura 1996). These inflammatory molecules may have local effects on white adipose tissue physiology as well as potential systemic effects on other organs, which can culminate in insulin resistance (Kershaw 2004). In addition, elevated levels of acute-phase reactants, such as TNF-α, IL-6, and C-reactive protein, and decreased levels of the adipose-specific secretory proteins, such as adiponectin, are highly correlated with cardiovascular problems (Kaplan 2001, Libby 2002, Matsuda 2002, and Ouchi 2001). Elevated levels of inflammatory mediators are also associated with insulin resistance and type II diabetes (Pickup 1997 and 2000). Further, it has been shown that TNF-α secreted from adipocytes mediates insulin resistance in an autocrine fashion (Engleman 2000 and Hotamisligil 1993 and 1994).

The adipocyte exerts an important role in energy homeostasis, both as depot for energy-rich triglycerides and as a source for metabolic hormones. Adipocytes also contribute to inflammation and the innate immune response. Although it can be physiologically beneficial to combine these two functions in a single cell type under some circumstances, the pro-inflammatory signals emanating from adipocytes in an obese state can have local and systemic effects that promote atherosclerosis and insulin resistance. The adipocyte displays a high level of sensitivity to bacterial lipopolysaccharide (LPS), TNF-α, IL-6, interferon-γ, and a host of other factors. Activation of NF-κB by TNF-α was shown to cause de-differentiation of adipocytes in culture, an effect specifically antagonized by the adipogenic transcription factor, peroxisome proliferator-activated receptor (PPAR)γ, and mediated by its newfound ability to override the inhibitory effects of NF-κB on the expression of key adipocyte genes (Ruan 1999 and 2003). In addition, TNF-α and LPS both induce expression and activity of inducible nitric oxide synthase (iNOS), a downstream target of NF-κB transcription (Kapur 1999). Adipose tissue iNOS induction has been observed in the obese state, and iNOS-deficient mice are partially protected from obesity-induced insulin resistance and glucose intolerance (Perreault 2001). Together, these findings implicate NF-κB signaling as a molecular link between inflammation and metabolic dysregulation in the adipocyte.

Accordingly, there is a need for compositions and methods to interfere with NF-κB signaling cascades for the treatment of obstructive respiratory conditions, metabolic syndrome and related diseases, such as IGFBP-3 receptor agonists. It is to the provision of such compositions and methods to interfere with NF-κB signaling cascades for the treatment of obstructive respiratory conditions, metabolic syndrome, cancer and related diseases that the various embodiments of the present invention are directed.

### BRIEF SUMMARY OF THE INVENTION

Aspects of the present invention are directed to methods and compositions for the treatment of metabolic syndrome, obstructive respiratory disorders, cancer and related diseases. Various embodiments described herein are compositions comprising IGFBP-3 receptor agonists and methods for the treatment of obstructive respiratory disorders, metabolic syndrome, cancer and related diseases with IGFBP-3 receptor agonists.

An obstructive respiratory disorder may be acute or chronic. In certain embodiments an acute obstructive respiratory disorder is an allergic reaction, such as hypersensitivity pneumonitis, or temporary asthma-like symptoms, such as the asthma like symptoms seen in certain subjects with metabolic syndrome. In certain embodiments a chronic obstructive respiratory disorder may be a chronic obstructive pulmonary disease (COPD), which may include asthma, cystic fibrosis, chronic bronchitis, emphysema, or bronchiectasis.

One aspect described herein is a method for interfering with the activity of nuclear factor-kappaB (NF-κB) in a cell, comprising providing to a cell an effective amount of a composition comprising an IGFBP-3 receptor agonist. In one embodiment of the present invention, a method for interfering with the activity of NF-κB in a cell can further comprise interfering with an activity of NF-κB in the cell, and interfering with an activity of NF-κB can comprises interfering with a NF-κB signaling pathway. In one embodiment, the IGFBP-3 receptor agonist comprises at least a portion of IGFBP-3. In another embodiment, the IGFBP-3 receptor agonist comprises an antibody or a fragment thereof capable of binding at least a portion of an IGFBP-3 receptor. In yet another embodiment, the composition comprises a vector capable of expressing at least a portion of IGFBP-3. The vector can be an adenovirus expressing at least a portion of IGFBP-3. The cell may be a cell involved in an obstructive respiratory disorder. The cell may be an adipocyte. In some embodiments, providing to a cell an effective amount comprises providing from about 0.001 μg to about 1,000 mg/kg subject/day. In another exemplary embodiment of the present invention, the composition may comprise a cell genetically engineered to overexpress at least a portion of IGFBP-3. In certain embodiments, the cell may be a mesenchymal stem cell.

Another aspect of the present invention comprises a method for decreasing insulin resistance of a cell, comprising: providing to a cell having insulin resistance an effective amount of a composition comprising an IGFBP-3 receptor agonist; and decreasing insulin resistance of the cell. A method for decreasing insulin resistance of a cell can further comprise increasing uptake of glucose by the cell, and increasing uptake of glucose by the cell can comprise increasing uptake of glucose by a cell by about at least 100% as compared to a cell not provided with an effective amount of a composition comprising an IGFBP-3 receptor agonist. In one embodiment of the present invention, decreasing insulin resistance of the cell can comprise interfering with an NF-κB signaling pathway. The IGFBP-3 receptor agonist may comprise many compounds including, but not limited to, IGFBP-3, a portion of IGFBP-3, a receptor agonist antibody or a fragment thereof which is capable of binding at least a portion of an IGFBP-3 receptor, or a vector capable of expressing at least a portion of IGFBP-3. In certain embodiments, the IGFBP-3 receptor agonist is a receptor agonist antibody or a fragment thereof which is capable of binding at least a portion of an IGFBP-3 receptor. In one embodiment, the vector can comprise an adenovirus expressing at least a portion of IGFBP-3. In some methods, the cell can be an adipocyte. A method for decreasing insulin resistance of a cell can involve providing from about 0.001 μg to about 1,000 mg/kg subject/day of a composition to a cell. In another embodiment of the present invention, the composition can comprise a cell genetically engineered to overexpress at least a portion of IGFBP-3. In one embodiment, the cell is a mesenchymal stem cell.

Another aspect of the present invention comprises a method for reducing expression of monocyte chemoattractant protein-1 (MCP-1) in a cell, comprising: providing to a cell an effective amount of a composition comprising an IGFBP-3 receptor agonist; and reducing expression of MCP-1 in the cell. The IGFBP-3 receptor agonist may comprise many compounds including, but not limited to, IGFBP-3, a portion of IGFBP-3, a receptor agonist antibody or a fragment thereof which is capable of binding at least a portion of an IGFBP-3 receptor, or a vector capable of expressing at least a portion of IGFBP-3. In certain embodiments, the IGFBP-3 receptor agonist is a receptor agonist antibody or a fragment thereof which is capable of binding at least a portion of an IGFBP-3 receptor. In one embodiment, the vector comprises an adenovirus expressing at least a portion of IGFBP-3. In one embodiment, the cell is an adipocyte. In some embodiment, providing to a cell an effective amount can comprise providing from about 0.001 μg to about 1,000 mg/kg subject/day of a composition. In one embodiment, a composition can comprise a cell genetically engineered to overexpress at least a portion of IGFBP-3, and the cell can be a mesenchymal stem cell.

In one embodiment there is provided a method of treating an obstructive respiratory disorder, comprising administering to a subject having an obstructive respiratory disorder a therapeutically effective amount of an IGFBP-3 receptor agonist. The IGFBP-3 receptor agonist may comprise many compounds including, but not limited to, IGFBP-3, a portion of IGFBP-3, a receptor agonist antibody or a fragment thereof which is capable of binding at least a portion of an IGFBP-3 receptor, or a vector capable of expressing at least a portion of IGFBP-3. In certain embodiments, the IGFBP-3 receptor agonist is a receptor agonist antibody or a fragment thereof which is capable of binding at least a portion of an IGFBP-3 receptor.

In the embodiments described herein, the antibody may be a monoclonal antibody. In other embodiments described herein the antibody may be a polyclonal antibody. In one embodiment, the vector comprises an adenovirus expressing at least a portion of IGFBP-3. In some embodiments, administering to a subject a therapeutically effective amount of an IGFBP-3 receptor agonist administering from about 0.001 μg to about 1,000 mg/kg subject/day of the agonist.

In yet another aspect there is provided a method for treating a metabolic syndrome, comprising administering to a subject having a metabolic syndrome a therapeutically effective amount of a composition comprising an IGFBP-3 receptor agonist. The IGFBP-3 receptor agonist may comprise many compounds including, but not limited to, IGFBP-3, a portion of IGFBP-3, a receptor agonist antibody or a fragment thereof which is capable of binding at least a portion of an IGFBP-3 receptor, or a vector capable of expressing at least a portion of IGFBP-3. In certain embodiments, the IGFBP-3 receptor agonist is a receptor agonist antibody or a fragment thereof which is capable of binding at least a portion of an IGFBP-3 receptor. In one embodiment, the vector comprises an adenovirus expressing at least a portion of IGFBP-3. In some embodiments, administering to a subject a therapeutically effective amount of a composition comprises administering from about 0.001 μg to about 1,000 mg/kg subject/day of the composition.

In one embodiment, the metabolic syndrome is insulin resistance. In such embodiments, a method for treating a metabolic syndrome can further comprise decreasing insulin resistance of the subject and increasing uptake of glucose by the subject. Increasing uptake of glucose by the subject can comprise increasing uptake of glucose by the subject by about at least 100% as compared to a subject having a metabolic syndrome comprising insulin resistance not provided with an effective amount of a composition comprising an IGFBP-3 receptor agonist.

In another embodiment, the metabolic syndrome is atherosclerosis. In such an embodiment, a method for treating a metabolic syndrome can further comprise reducing the expression of MCP-1 in the subject.

In yet another embodiment, the method for treating a metabolic syndrome may comprise administering to a subject having a metabolic syndrome a therapeutically effective amount of a composition comprising an IGFBP-3 receptor agonist, wherein the composition comprises a cell genetically engineered to overexpress at least a portion of IGFBP-3. In such an embodiment, the cell may be a mesenchymal stem cell.

An additional embodiment includes methods for treating cancer, comprising: administering to a subject having cancer cells a therapeutically effective amount of a composition comprising an IGFBP-3 receptor agonist. The IGFBP-3 receptor agonist may comprise many compounds including, but not limited to, IGFBP-3, a portion of IGFBP-3, a receptor agonist antibody or a fragment thereof which is capable of binding at least a portion of an IGFBP-3 receptor, or a vector capable of expressing at least a portion of IGFBP-3. In certain embodiments, the IGFBP-3 receptor agonist is a receptor agonist antibody or a fragment thereof which is capable of binding at least a portion of an IGFBP-3 receptor. In one embodiment, the vector comprises an adenovirus expressing at least a portion of IGFBP-3. In some embodiments, administering to a subject a therapeutically effective amount of a composition comprises administering from about 0.001 μg to about 1,000 mg/kg subject/day of the composition.

Other aspects and features of embodiments will become apparent to those of ordinary skill in the art, upon reviewing the following description of specific, exemplary embodiments of described herein in conjunction with the accompanying figures.

### BRIEF DESCRIPTION OF DRAWINGS

FIG. 1 shows adipocyte differentiation in human adipocytes.

FIG. 2A is a Western blot demonstrating the effect of TNF-α on NF-κB signaling in adipocytes.

FIG. 2B is a Western blot showing the effect of TNF-α on IRS-1 expression in adipocytes.

FIG. 3A is an agarose gel of RT-PCR products of adipocytes treated with TNF-α and an adenoviral vector expressing IGFBP-3.

FIG. 3B is an agarose gel of RT-PCR products of adipocytes treated with TNF-α and an adenoviral vector expressing IGFBP-3.

FIG. 3C is a Western blot of adipocytes treated with TNF-α and an adenoviral vector expressing IGFBP-3.

FIG. 4A is an agarose gel of RT-PCR products of adipocytes treated with TNF-α and an adenoviral vector expressing an IGFBP-3 mutant, IGFBP-3^{GGG}.

FIG. 4B is a Western blot of adipocytes treated with TNF-α and an adenoviral vector expressing an IGFBP-3 mutant, IGFBP-3^{GGG}.

FIG. 5A graphically depicts the effect of IGFBP-3 on TNF-α-induced insulin resistance in human adipocytes.

FIG. 5B graphically depicts the effect of IGFBP-3 on TNF-α-induced glucose uptake in human adipocytes.

FIG. 5C graphically depicts the effect of IGFBP-3 on TNF-α-induced glucose uptake in murine 3T3 adipocytes.

FIG. 6A graphically depicts the effect of IGFBP-3 and antibodies specific for the IGFBP-3 receptor on TNF-α-induced glucose uptake in human adipocytes.

FIG. 6B graphically depicts the effect of IGFBP-3 and antibodies specific for the IGFBP-3 receptor on TNF-α-induced glucose uptake in murine 3T3 adipocytes.

FIG. 7 compares the effects of IGFBP-3 to rosiglitazone on TNF-α-regulated proteins.

FIG. 8 graphically depicts that treatment of a subject with 5 ug/ml of the purified IGFBP-3R agonistic antibody resulted in a complete suppression of TNF-α-induced ICAM-1 expression as well as a decrease of IκBα and p65-NF-κB expression.

FIG. 9 is a schematic diagram of the experimental protocol. Mice were sensitized on days 1 and 14 by intraperitoneal injection of OVA emulsified in 1 mg of aluminum hydroxide. On days 21, 22, and 23 after the initial sensitization, the mice were challenged for 30 minutes with an aerosol of 3% (w/v) OVA in saline (or with saline as a control) using an ultrasonic nebulizer. In the case of treatment with Ad vector, it was administered intratracheally two times to each treated animal, once on day 21 (1 hour before the first airway challenge with OVA) and the second time on day 23 (3 hours after the last airway challenge with OVA).

FIGS. 10A and 10B graphically depicts data demonstrating the induction of apoptosis by agonistic IGFBP-3R antibodies; FIG. 10A shows data demonstrating that treatment with polyclonal IGFBP-3R antibodies, but not preimmune sera, resulted in induction of apoptosis in human prostate cancer cells and FIG. 10B shows data that demonstrates that the potency of the agonistic antibodies for induction of apoptosis is comparable with that of IGFBP-3.

### DETAILED DESCRIPTION OF THE INVENTION

The phrase “obstructive respiratory disorder” as used herein refers to conditions associated with airway obstruction. This obstruction may arise from airway hyperresponsiveness, inflammation of the respiratory tissue, thickening of the respiratory tissue, or any combination of two or more these. In one embodiment, the affected respiratory tissue is lower respiratory tissue. An obstructive respiratory disorder may be either acute or chronic. Acute disorders include allergic reactions and temporary asthma-like symptoms. Chronic disorders include chronic obstructive pulmonary diseases (COPDs), which may include asthma, cystic fibrosis, chronic bronchitis, emphysema, or bronchiectasis.

The insulin-like growth factor (IGF) system is a multi-component network of molecules that is ubiquitously involved in the regulation of growth, proliferation, and differentiation of a variety of cell types. IGFs are capable of stimulating tissue growth and differentiation by acting in a paracrine, autocrine, and/or endocrine manner. The mitogenic actions of IGFs are mediated largely through the type I IGF receptor (IGFR-I), which is a heterotetrameric, membrane-spanning tyrosine kinase. IGFR-I binds both IGF-I and IGF-II with high affinity, and binds insulin with a substantially lower affinity. Insulin-like growth factors binding proteins (IGFBP), numbered 1 through 6, bind IGF-I and IGF-II with high affinity. IGF-1 activity is modulated by IGFBPs. IGFBPs play a role in transporting IGFs, prolonging their half-lives by protecting them from proteolytic degradation, and regulating their availability for interaction with IGFRs. In this manner, they modulate the effects of IGF on growth and differentiation by either potentiating or inhibiting IGF activity. Further, IGF-I appears to be involved in the inflammatory processes.

Although the six IGFBPs display high levels of conservation in their C- and N-terminal domains, their expression patterns and properties vary widely. Recent research has demonstrated that IGFBPs have unique intrinsic biological activities beyond their ability to interact with IGF, which are termed the “IGF-independent” actions. For example, IGFBP-3 has been shown to exert IGF-independent effects on cell growth and apoptosis. Despite this work, the mechanism underlying the IGF-independent actions of IGFBP-3 has yet to be fully elucidated. Further, the pathophysiological role of IGFBP-3 in inflammation is unknown. The present invention unexpectedly demonstrates that both wild-type IGFBP-3 and the GGG-IGFBP-3 mutant are potent inhibitors of inflammation and metabolic dysregulation.

One aspect of the present invention comprises pharmaceutical compositions comprising an IGFBP-3 receptor agonist. As used herein, the term “receptor agonist” refers to a ligand or agent which may bind or associate with a receptor to alter the activity of a receptor. A receptor agonist can be distinguished from an antagonist, which is a type of ligand or agent that may also bind or associate with a receptor, but does not alter the activity of the receptor. An IGFBP-3 receptor agonist can comprise a direct IGFBP-3 receptor agonist or indirect an IGFBP-3 receptor agonist. In one embodiment of the present invention, an IGFBP-3 receptor agonist may be able to directly bind or associate with the IGFBP-3 receptor. In another embodiment, an IGFBP-3 receptor agonist may be able to indirectly alter the activity of the IGFBP-3 receptor by exerting an effect on the IGFBP-3 signaling cascade. In yet another embodiment, an IGFBP-3 receptor agonist may be able to indirectly alter the activity of the IGFBP-3 receptor by increasing the production of an IGFBP-3 receptor agonist, such as IGFBP-3.

An IGFBP-3 receptor agonist may be selected from amongst many biological or chemical compounds, including, but not limited to, a simple or complex organic or inorganic molecule, peptide, peptide mimetic, a protein (e.g. antibody or growth factor), an antigen or immunogen, a polynucleotide (e.g., a microRNA, siRNA), a virus, or a therapeutic agent. Organic or inorganic molecules can include, but are not limited to, a homogenous or heterogeneous mixture of compounds, including pharmaceuticals, radioisotopes, crude or purified plant extracts, and/or an entity that alters, inhibits, activates, or otherwise affects biological or biochemical events, including classes of molecules (e.g., proteins, amino acids, peptides, polynucleotides, nucleotides, carbohydrates, sugars, lipids, nucleoproteins, glycoproteins, lipoproteins, steroids, growth factors, chemoattractants, cytokines, chemokines, etc.) that are commonly found in cells and tissues, whether the molecules themselves are naturally-occurring or artificially created (e.g., by synthetic or recombinant methods). A compound may also comprise one or more pharmaceutical additives including, but not limited to, solubilizers, emulsifiers, buffers, preservatives, suspending agents, thickening agents, stabilizers, inert components, and the like.

Examples of such compounds include, but are not limited to, agents for gene therapy; analgesics; anti-arthritics; anti-asthmatic agents; anti-cancer agents; anti-cholinergics; anti-convulsants; anti-depressants; anti-diabetic agents; anesthetics; antibiotics; antigens; anti-histamines; anti-infectives; anti-inflammatory agents; anti-microbial agents; anti-fungal agents, anti-Parkinson agents; anti-spasmodics; anti-pruritics; anti-psychotics; anti-pyretics; anti-viral agents; nucleic acids; DNA; RNA; polynucleotides; nucleosides; nucleotides; amino acids; peptides; proteins; carbohydrates; lectins; lipids; fats; fatty acids; viruses; immunogens; antibodies and fragments thereof, including but not necessarily limited to monoclonal antibodies and polyclonal antibodies and antigen-binding fragments thereof; sera; immunostimulants; immunosuprressants; cardiovascular agents; channel blockers (e.g., potassium channel blockers, calcium channel blockers, beta-blockers, alpha-blockers); anti-arrhythmics; anti-hypertensives; inhibitors of DNA, RNA, or protein synthesis; neurotoxins; vasodilating agents; vasoconstricting agents; gases, growth factors; growth inhibitors; hormones; steroids; steroidal and non-steroidal anti-inflammatory agents; corticosteroids; angiogenic agents; anti-angiogenic agents; hypnotics; muscle relaxants; muscle contractants; sedatives; tranquilizers; ionized and non-ionized active agents; metals; small molecules; pharmaceuticals; hemotherapeutic agents; wound healing agents; indicators of change in the bio-environment; enzymes; enzyme inhibitors; nutrients; vitamins; minerals; coagulation factors; anticoagulants; anti-thrombotic agents; neurochemicals (e.g., neurotransmitters); cellular receptors; radioactive materials; contrast agents (e.g., fluorescence, magnetic, radioactive); nanoparticles; vaccines; modulators of cell growth; modulators of cell adhesion; cell response modifiers; cells; chemical or biological materials or compounds that induce a desired biological or pharmacological effect; and combinations thereof.

In an exemplary embodiment of the present invention, an IGFBP-3 receptor agonist can comprises at least a portion of IGFBP-3. For example, an IGFBP-3 receptor agonist can comprise the entire IGFBP-3 protein or a portion of IGFBP-3 protein (e.g., a peptide, polypeptide) capable of engaging an IGFBP-3 receptor. In an exemplary embodiment of the present invention, the IGFBP-3 is human IGFBP-3 or a homolog (e.g., mammalian homologs) thereof having substantial or complete identity to human IGFBP-3. As used herein, the term “substantial identity” of nucleotide sequence or protein sequence means that a nucleotide sequence or protein sequence includes a sequence that has at least 51%, 52%, 53%, 54%, 55%, 56%, 57%, 58%, 59%, 60%, 61%, 62%, 63%, 64%, 65%, 66%, 67%, 68%, or 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence (e.g., human IGFBP-3).

In yet another exemplary embodiment, the IGFBP-3 receptor agonist is an antibody or a fragment thereof which is capable of specifically binding to at least a portion of an IGFBP-3 receptor. As used herein, the term “antibody” includes intact monoclonal and polyclonal antibody molecules, as well as antibody fragments (such as, for example, Fab and F(ab′)2 fragments). Fab and F(ab′)2 fragments lack the Fc fragment of an intact antibody, clear more rapidly from the circulation, and may have less non-specific tissue binding than an intact antibody. Agonist antibodies or fragments thereof will, when binding to the IGFBP-3 receptor, cause the receptor to increase at least one signaling activity that is increased upon interaction with IGFBP-3. Antibodies of the present invention may be humanized or not, and may have functional groups or tags associated with them for monitoring functions or for providing additional activities or functionalities.

Native antibodies are an important part of the immune system and have unique Y-shaped structures that may bind antigens. Each end of an antibody has a specific paratope that binds with a complementary epitope of the antigen. With this mechanism, the antibody can identify the antigen as a foreign structure for attack by other components of the immune system. Full-length antibodies, as they exists naturally, are immunoglobulin molecules comprising four peptide chains, two heavy (H) chains (about 50-70 kDa when full length) and two light (L) chains (about 25 kDa when full length) interconnected by disulfide bonds. The amino terminal portion of each chain includes a variable region of about 100-110 or more amino acids primarily responsible for antigen recognition.

The carboxy-terminal portion of each chain defines a constant region primarily responsible for effector function. Light chains are classified as kappa or lambda and characterized by a particular constant region. Each light chain is comprised of an N-terminal light chain variable region (herein “LCVR”) and a light chain constant region comprised of one domain, CL. Heavy chains are classified as gamma, mu, alpha, delta, or epsilon, and define the antibody's isotype as IgG, IgM, IgA, IgD, and IgE, respectively and several of these may be further divided into subclasses (isotypes) e.g., IgG1, IgG2, IgG3, IgG4, IgA1 and IgA2. Each heavy chain type is characterized by a particular constant region. Each heavy chain is comprised of an N-terminal heavy chain variable region (herein “HCVR”) and a heavy chain constant region. The heavy chain constant region is comprised of three domains (CH1, CH2, and CH3) for IgG, IgD, and IgA; and 4 domains (CH1, CH2, CH3, and CH4) for IgM and IgE.

The HCVR and LCVR regions can be further subdivided into regions of hypervariability, termed complementarity determining regions (“CDRs”), interspersed with regions that are more conserved, termed framework regions CFR”). Each HCVR and LCVR is composed of three CDRs and four FRs, arranged from amino-terminus to carboxy-terminus in the following order: FR1, CDR1, FR2, CDR2, FR3, CDR3, FR4.

The variable region of each light-heavy chain pair forms an antigen-binding site of the antibody. Thus, an intact IgG antibody has two antigen-binding sites. Except in bifunctional or bispecific antibodies, the two antigen-binding sites of the antibody are the same. As used herein, the “antigen-binding portion” or “antigen-binding region” or “antigen-binding domain” refers interchangeably to that portion of an antibody

For the purposes of the present description, a “monoclonal antibody” as used herein refers to a rodent, preferably murine antibody, a chimeric antibody, a humanized antibody or a fully human antibody, unless otherwise indicated herein. The term “monoclonal antibody” as used herein is not limited to antibodies produced through hybridoma technology. For the purposes of the present description, a “monoclonal antibody” refers to an antibody that is derived from a single copy or clone, including e.g., eukaryotic, prokaryotic, or phage clone, and not the method by which it is produced.

Preparation of immunogenic antigens and monoclonal antibody production can be performed using any suitable technique. A variety of methods have been described (see e.g., Kohler et al. 1975 Nature 256 495-7 and Kohler et al. 1976 Eur J Immunol 6 511-9; Galfre et al. 1977 Nature 266 550-2; Koprowski et al., U.S. Pat. No. 4,172,124; Harlow, E. and D. Lane, 1988, Using Antibodies: A Laboratory Manual, Cold Spring Harbor Laboratory: Cold Spring Harbor, N.Y.; Current Protocols In Molecular Biology, Vol. 2 (e.g., Supplement 27, Summer '94), Ausubel, F. M. et al., Eds., John Wiley & Sons: New York, N.Y., Chapter 11, (1991-2003)), each of which is entirely incorporated herein by reference.

Generally, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromyelomas, fusion products thereof, or any cell or fusion cell derived therefrom, or any other suitable cell line as known in the art, see, e.g., www.atcc.org, www.lifetech.com., and the like, each of which is entirely incorporated herein by reference) with antibody producing cells, such as, but not limited to, isolated or cloned spleen cells, or any other cells expressing heavy or light chain constant, variable, framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, each entirely incorporated herein by reference.

Antibody producing cells can be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge Antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 35 08/350,260 (May 12, 1994); PCT/GB94/01422; PCT/GB94/02662; PCT/GB97/01835; (CAT/MRC); WO90/14443; WO90/14424; WO90/14430; PCT/U5594/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), each entirely incorporated herein by reference) or that rely upon immunisation of transgenic animals (e.g., SCID mice, Nguyen et al. 1997 Microbiol Immunol 41 901-7; Sandhu et al. 1996 Crit Rev Biotechnol 16 95-118; each entirely incorporated by reference as well as related patents and applications) that are capable of producing a repertoire of human antibodies, as known in the art and/or as described herein. Such techniques, include, but are not limited to, ribosome display (Hanes et al. 1997 Proc Natl Acad Sci USA 94 4937-42; Hanes et al. 1998 Proc Natl Acad Sci USA 95 14130-5); single cell antibody producing technologies (e.g., selected lymphocyte antibody method (“SLAM”) U.S. Pat. No. 5,627,052, Wen et al. 1987 Eur J Immunol 17 887-92; Babcook et al. 1996 Proc Natl Acad Sci USA 93 7843-8); gel microdroplet and flow cytometry (Powell et al. 1990 Biotechnology (N Y) δ 333-7); One Cell Systems, Cambridge, Mass.; (Gray et al. 1995 J Immunol Methods 182 155-63; Kenney et al. 1995 Biotechnology (NY) 13 787-90); B-cell selection (Steenbakkers et al. 1994 Mol Biol Rep 19 125-34; Jonak et al., Progress Biotech, Vol. 5, In Vitro Immunization in Hybridoma Technology, Borrebaeck, ed., Elsevier Science Publishers B.V., Amsterdam, Netherlands (1988), each of which is entirely incorporated herein by reference).

A “monoclonal antibody” can be an intact antibody (comprising a complete or full-length Fc region), a substantially intact antibody, or a portion or fragment of an antibody comprising an antigen-binding portion, e.g., a Fab fragment, Fab′ fragment or F(ab′)2 fragment of a murine antibody or of a chimeric, humanized or human antibody. The “Fab” fragment contains a variable and constant domain of the light chain and a variable domain and the first constant domain (CHI) of the heavy chain. “F(ab′),” antibody fragments comprise a pair of Fab fragments which are generally covalently linked near their carboxy termini by hinge cysteines between them. Other chemical couplings of antibody fragments are also known in the art.

For in vivo use of antibodies in humans, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison S L 1985 Science. September 20; 229(4719):1202-7; Oi 1986 BioTechniques 4:214; Gillies S D, Lo K M, Wesolowski J 1989 J Immunol Methods. December 20; 125(1-2):191-202; U.S. Pat. Nos. 5,807,715; 4,816,567; and 4,816,397.

Included within the scope of the invention, and useful in practicing the methods of the invention, are de-immunized antibodies that have sequence variations produced using methods described in, for example, Patent Publication Nos. EP 0983303A1, WO 2000/34317, and WO 98/52976.

Another approach included within the scope of the invention in order to minimize the immunogenic and allergic responses intrinsic to mouse or other non-human monoclonal antibodies and thus to increase the efficacy and safety of the administered antibodies, is “veneering”. The term “veneered antibody” refers to the selective replacement of framework region residues from, for example, a mouse heavy or light chain variable region with human framework region residues in order to provide a xenogeneic molecule comprising an antigen-binding site which retains substantially all of the native framework region folding structure. Veneering techniques are based on the understanding that the ligand-binding characteristics of an antigen-binding site are determined primarily by the structure and relative disposition of the heavy and light chain CDR sets within the antigen-binding surface. Thus, antigen-binding specificity can be preserved in a humanized antibody only wherein the CDR structures, their interaction with each other, and their interaction with the rest of the V region domains are carefully maintained. By using veneering techniques, exterior (e.g. solvent accessible) framework region residues, which are readily encountered by the immune system, are selectively replaced with human residues to provide a hybrid molecule that comprises either a weakly immunogenic, or substantially non-immunogenic, veneered surface.

The scope of the present invention also extends to humanized agonist antibodies to IGFBP-3R. By “humanized” is intended forms of agonist antibodies to IGFBP-3R that contain minimal sequence derived from non-human immunoglobulin sequences. For the most part, humanized antibodies are human immunoglobulins (recipient antibody) in which residues from a hypervariable region (also known as complementarity determining region or CDR) of the recipient are replaced by residues from a hypervariable region of a non-human species (donor antibody) such as mouse, rat, rabbit, or nonhuman primate having the desired specificity, affinity, and capacity.

Humanized antibodies within the scope, and suitable for use in the methods, of the present invention may, for example, have binding characteristics similar to those exhibited by non-humanized antibodies.

Humanization can be essentially performed following the method of Winter and co-workers (Jones P T, Dear P H, Foote J, Neuberger M S, Winter G 1986 Nature. May 29-June 4; 321(6069):522-5; Riechmann L, Clark M, Waldmann H, Winter G 1988 Nature. March 24; 332(6162):323-7; Verhoeyen M, Milstein C, Winter G 1988 Science. March 25; 239(4847):1534-6), by substituting rodent or mutant rodent CDRs or CDR sequences for the corresponding sequences of a human antibody. See also U.S. Pat. Nos. 5,225,539; 5,585,089; 5,693,761; 5,693,762; and 5,859,205.

Antibodies can be humanized using a variety of techniques known in the art including, for example, CDR-grafting (EP 239400; PCT publication WO 91/09967; U.S. Pat. Nos. 5,225,539; 5,530,101; and 5,585,089), veneering or resurfacing (EP 592106; EP 519596; Padlan E A 1991 Mol. Immunol. April-May; 28(4-5):489-98; Studnicka G M, Soares S, Better M, Williams R E, Nadell R, Horwitz A H 1994 Protein Eng. June; 7(6):805-14; Roguska M A, Pedersen J T, Keddy C A, Henry A H, Searle S J, Lambert J M, Goldmacher V S, Blattler W A, Rees A R, Guild B C 1994 Proc Natl Acad Sci USA. February 1; 91(3):969-73), and chain shuffling (U.S. Pat. No. 5,565,332).

In some instances, residues within the framework regions of one or more variable regions of the human immunoglobulin are replaced by corresponding non-human residues (see, for example, Queen et al. U.S. Pat. No. 5,585,089; U.S. Pat. Nos. 5,693,761; 5,693,762; and 6,180,370; see also, e.g., Riechmann (1988)).

“Superhumanization” is a humanization approach where the CDRs conferring antigen specificity (‘donor’) are grafted to human germline framework sequences (‘acceptor’) that are known to be expressed with human CDRs that are structurally identical or similar to the ‘donor’ CDRs (Tan P, Mitchell D A, Buss T N, Holmes M A, Anasetti C, Foote J 2002 J Immunol. July 15; 169(2):1119-25, see also International Publication No. WO 2004/006955). By using frameworks encoded by human genomic V gene sequences, rather than sequences that can include somatic mutations, this approach has enhanced potential for reduced immunogenicity. By emphasizing the structural homologies between donor and acceptor CDRs, this approach also has enhanced potential for affinity retention.

Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence.

The humanized antibody optionally also will comprise at least a portion of an immunoglobulin constant region (Fc), typically that of a human immunoglobulin. For further details see Jones P T, Dear P H, Foote J, Neuberger M S, Winter G 1986 Nature. May 29-June 4; 321(6069):522-5; Riechmann L, Clark M, Waldmann H, Winter G 1988 Nature. March 24; 332(6162):323-7; Presta 1992. Curr Opin Struct Biol. 2:593-596. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 2001/27160 where humanized antibodies and techniques for producing humanized antibodies having improved affinity for a predetermined antigen are disclosed.

Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al. 1988. Biotechnology 12:899-903).

Human antibodies can also be produced using transgenic animals which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes.

For an overview of this technology for producing human antibodies, see Lonberg N, Huszar D 1995 Int Rev Immunol. 1995; 13(1):65-93. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0598877; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598.

The therapeutic utility of antibodies can be enhanced by modulating their functional characteristics, such as serum half-life, biodistribution and binding to Fc receptors. This modulation can be achieved by protein engineering, glycoengineering or chemical methods. Depending on the therapeutic application and the desired level of effector activity required, it could be advantageous to either increase or decrease any of these activities.

A number of methods for modulating antibody serum half-life and biodistribution are based on modifying the interaction between antibody and the neonatal Fc receptor (FcRn), a receptor with a key role in protecting IgG from catabolism, and maintaining high serum antibody concentration. Dall'Acqua Dall'Acqua W F, Kiener P A, Wu H 2006 J Biol Chem. August 18; 281(33):23514-24. Epub 2006 Jun. 21 describe substitutions in the Fc region of IgG1 that enhance binding affinity to FcRn, thereby increasing serum half-life, and further demonstrate enhanced bioavailability and modulation of ADCC activity with triple substitution of M252Y/S254T/T256E. See also U.S. Pat. Nos. 6,277,375; 6,821,505; and 7,083,784. Hinton et al (Hinton P R, Johlfs M G, Xiong J M, Hanestad K, Ong K C, Bullock C, Keller S, Tang M T, Tso J Y, Vásquez M, Tsurushita N 2004 J Biol. Chem. February 20; 279(8):6213-6. Epub 2003 Dec. 29 and Hinton P R, Xiong J M, Johlfs M G, Tang M T, Keller S, Tsurushita N 2006 J. Immunol. January 1; 176(1):346-56), have described constant domain amino acid substitutions at positions 250 and 428 that confer increased in vivo half-life. See also U.S. Pat. No. 7,217,797. Petkova et al (Petkova S B, Akilesh S, Sproule T J, Christianson G J, Al Khabbaz H, Brown A C, Presta L G, Meng Y G, Roopenian D C 2006 Int Immunol. December; 18(12):1759-69. Epub 2006 Oct. 31) have described constant domain amino acid substitutions at positions 307, 380 and 434 that confer increased in vivo half-life. See also Shields et al (Shields R L, Namenuk A K, Hong K, Meng Y G, Rae J, Briggs J, Xie D, Lai J, Stadlen A, Li B, Fox J A, Presta L G 2001 J Biol Chem. March 2; 276(9):6591-604. Epub 2000 Nov. 28) and WO 2000/42072. Other examples of constant domain amino acid substitutions which modulate binding to Fc receptors and subsequent function mediated by these receptors, including FcRn binding and serum half-life, are described in U.S. Pat. Application Nos 20090142340; 20090068175; and 20090092599.

The glycans linked to antibody molecules are known to influence interactions of antibody with Fc receptors and glycan receptors and thereby influence antibody activity, including serum half-life (Kaneko Y et al, (Kaneko Y, Nimmerjahn F, Ravetch J V 2006 Science. August 4; 313(5787):670-3); Jones A J et al, (Jones A J, Papac D I, Chin E H, Keck R, Baughman S A, Lin Y S, Kneer J, Battersby J E 2007 Glycobiology. May; 17(5):529-40. Epub 2007 Mar. 1); Kanda Y et al, (Kanda Y, Yamada T, Mori K, Okazaki A, Inoue M, Kitajima-Miyama K, Kuni-Kamochi R, Nakano R, Yano K, Kakita S, Shitara K, Satoh M 2007 Glycobiology. January; 17(1):104-18. Epub 2006 Sep. 29)). Hence, certain glycoforms that modulate desired antibody activities can confer therapeutic advantage. Methods for generating engineered glycoforms are known in the art and include but are not limited to those described in U.S. Pat. Nos. 6,602,684; 7,326,681; 7,388,081; and WO 2008/006554.

Extension of half-life by addition of polyethylene glycol (PEG) has been widely used to extend the serum half-life of proteins, as reviewed, for example, by Fishburn (Fishburn C S 2008 J Pharm Sci. October; 97(10):4167-83).

In order to generate IGFBP-3R agonist antibodies full cDNA sequence of IGFBP-3R [915 base pairs encoding a 240 amino acid polypeptide (GenBank accession #FJ748884)1 is employed to the suitable systems to generate antibodies described herein. A number of methods for screening IGFBP-3R agonist antibodies are based on ability of antibodies to specifically as well as preferentially bind to IGFBP-3R and exert subsequent biological functions which mimics those of IGFBP-3. Ingermann et al (Ingermann A R; Yang Y F; Han J; Mikami A; Garza A E; Mohanraj L; Fan L; Idowu M; Ware J L; Kim H S Lee D Y; Oh Y. J. Biol. Chem., 2010, 230(39): 30233-30246. October; 97(10):4167-83) describe an assay to screen specific interaction of peptides with IGFBP-3R in cell-free conditions. Screening monoclonal antibodies specific to IGFBP-3R can be achieved by modifying the assay. That is, recombinant FLAG-tagged IGFBP-3R overexpressed in COS-7 cell lysates are captured in 96-well plate coated with anti-FLAG antibody. Non-specific interaction of recombinant FLAG-tagged IGFBP-3R are blocked with 5% BSA and the plate is washed three times with PBS containing 0.2% Tween-20. After washing, biotinylated IGFBP-3 is incubated in the presence of various concentration of monoclonal antibody for 1 hour at room temperature. The wells are then incubated with HRP-conjugated Streptavidin diluted in HBSST-BSA for 1 hour at room temperature, and further incubated with 50 μl TMB substrate. The reaction is then terminated by adding 50 μl 1N H2SO4 and absorbance measured at 450 nm. Several functional assays will be employed to screen IGFBP-3R agonist antibodies based upon the previous description related to IGFBP-3 functional studies in a variety of human diseases. The agonistic behaviour of IGFBP-3R monoclonal antibody will be assayed and will be compared with that of IGFBP-3 using a cell death (apoptosis) assay in a variety of human cancer cells as described in EXAMPLE 7: Treatment of Cancer Cells with IGFBP-3R agonist antibodies and as described in Ingermann et al (supra). In brief, various concentration of each IGFBP-3R monoclonal antibody will be used to a variety of human cancer cell lines in culture for two days and apoptotic cell death or activation of caspases will be measured using a cell death detection ELISA and caspase activity assays.

In addition, anti-inflammatory behaviour of IGFBP-3R monoclonal antibody will be assayed using human differentiated adipocytes in vitro as described in EXAMPLES 1-3 and 5.

The term “specifically bind” as used herein refers to the situation in which one member of a specific binding pair does not significantly bind to molecules other than its specific binding partner(s). The term is also applicable where e.g., an antigen-binding domain of an antibody of the invention is specific for a particular epitope that is carried by a number of antigens, in which case the specific antibody carrying the antigen-binding domain will be able to bind to the various antigens carrying the epitope. Accordingly a monoclonal antibody of the invention specifically binds human IGFBP-3R. Further, a monoclonal antibody of the invention specifically binds human IGFBP-3R and cynomolgus monkey IGFBP-3R but does not specifically bind rat or murine IGFBP-3R. Further a monoclonal antibody of the invention specifically binds a non-linear or conformational human IGFBP-3R epitope.

The term “preferentially bind” as used herein, refers to the situation in which an antibody binds a specific antigen at least about 20% greater, preferably at least about 50%, 2-fold, 20-fold, 50-fold or 100-fold greater than it binds a different antigen as measured by a technique available in the art, e.g., competition ELISA or KD measurement with a BIACORE or KINEXA assay. An antibody may preferentially bind one epitope within an antigen over a different epitope within the same antigen. Accordingly an antibody of the invention preferentially binds human IGFBP-3R over rabbit IGFBP-3R.

In one exemplary embodiment of the present invention, the pharmaceutical composition comprises a vector capable of expressing an IGFBP-3 receptor agonist. The term “vector” as used herein refers to a vehicle into which a genetic element encoding a peptide or protein may be operably inserted so as to bring about the expression of that peptide or protein. Examples of suitable vectors include, but are not limited to, plasmids, phagemids, cosmids, artificial chromosomes, such as a yeast artificial chromosome (YAC), a bacterial artificial chromosome (BAC), or a P1-derived artificial chromosome (PAC), bacteriophages, such as lambda phage or M13 phage, and animal viruses. Animal viruses used as vectors can include, but are not limited to, a retrovirus (including lentivirus), an adenovirus, an adeno-associated virus, a herpesvirus (e.g., herpes simplex virus), a poxvirus, a baculovirus, a papillomavirus, and a papovavirus (e.g., SV40). A vector may contain a variety of elements for controlling expression of the peptide, including promoter sequences, transcription initiation sequences, enhancer sequences, selectable elements, and reporter genes. A vector may also include or be associated with various materials to aid in its entry into the cell, including but not limited to a virion, a liposome, or a protein coating.

In an exemplary embodiment of the present invention, a vector capable of expressing an IGFBP-3 receptor agonist comprises an adenovirus vector capable of expressing at least a portion of IGFBP-3. In another exemplary embodiment of the present invention, a vector capable of expressing an IGFBP-3 receptor agonist comprises an adenovirus vector capable of expressing IGFBP-3 or a mutant thereof. For example, an adenovirus vector may be capable of expressing wild-type IGFBP-3 or an IGFBP-3 mutant, such as an IGFBP-3 mutant that has no binding affinity for IGFs. An example of such an IGFBP-3 mutant is IGFBP-3^{GGG}, which is generated by site-directed mutagenesis of IGFBP-3 residues Ile⁵⁶, Leu⁸⁰, and Leu⁸¹ to Gly⁵⁶, Gly⁸⁰, and Gly⁸¹.

In yet another exemplary embodiment of the present invention, a pharmaceutical composition comprises a cell that expresses an IGFBP-3 receptor agonist. Such embodiments contemplate a cell that overexpresses an IGFBP-3 receptor agonist. As used herein, the term “overexpress” refers to any amount greater than or equal to an expression level exhibited by a reference standard. In one embodiment of the present invention, an adipocyte can be genetically engineered to overexpress an IGFBP-3 receptor agonist, such as IGFBP-3. In another embodiment of the present invention, a mesenchymal stem cell can be genetically engineered to overexpress an IGFBP-3 receptor agonist, such as IGFBP-3. In yet another embodiment of the present invention, an adipose tissue mesenchymal stem cell can be genetically engineered to overexpress an IGFBP-3 receptor agonist, such as IGFBP-3

An aspect of the present invention comprises a method for interfering with the activity of nuclear factor-kappaB (NF-κB), comprising, providing to a cell an effective amount of a composition comprising an IGFBP-3 receptor agonist; and interfering with the activity of NF-κB in the cell. NF-κB is a protein complex that acts a cellular transcription factor. NF-κB is found in almost all animal cell types and is involved in many cellular responses to stimuli, such as stress, cytokines, free radicals, ultraviolet irradiation, bacterial antigens, and viral antigens, among others. Further, NF-κB plays a key role in regulating the immune response, inflammation, cellular proliferation, and metabolic regulation of a cell. Consistent with this role, dysregulation of NF-κB has been linked to cancer, inflammatory diseases, autoimmune diseases, bacterial infection, viral infection, and improper immune system development. Thus, the compositions and methods of the present invention contemplate the provision of an IGFBP-3 receptor agonist to interfere with any NF-κB-related activity, including but not limited to, gene transcription and cellular signaling events.

As used herein, the phrase “interfering with the activity of NF-κB” can refer to both direct and indirect interference with the activity of the NF-κB protein, direct or indirect interference with the transcription of NF-κB genes or the translation of NF-κB mRNA, and direct and indirect interference with upstream and downstream effectors in the NF-κB signaling cascade. Furthermore, “interfering with the activity of NF-κB” can include partially interfering with the activity of NF-κB, substantially interfering with the activity of NF-κB, or completely interfering with the activity of NF-κB.

As used herein, the terms “interfering,” “preventing,” “reducing,” “altering,” or “inhibiting” refer to a difference in degree from a first state, such as an untreated state in a cell, to a second state, such as a treated state in a cell. For example, in the absence of treatment with the methods or compositions of the present invention, an NF-κB-related activity may occur at a first rate. If a cell is exposed to treatment with the methods or compositions of the present invention, the NF-κB-related activity occurs at a second rate, which is altered, lessened, or reduced from the first rate. Thus, the terms “interfering,” “preventing,” “reducing,” “altering,” or “inhibiting” may be used interchangeably through this application and may refer to a partial reduction, substantial reduction, near-complete reduction, complete reduction, or an absence of a NF-κB-related activity and the rate thereof.

The terms “subject,” “individual” or “cell” are used interchangeably herein, and refers to a vertebrate, preferably a mammal, and more preferably a human. Mammals include, but are not limited to, non-human primates, humans, cows, dogs, mice, rabbits, swine, rats, guinea pigs and equines. Tissues and cells are also encompassed by this terminology. In an exemplary embodiment of the present invention, a subject comprises a human. In an exemplary embodiment of the present invention, a subject comprises an adipocyte.

The term “an effective amount” in the context of the methods for interfering with the activity of NF-κB is considered to be any quantity of a IGFBP-3 receptor agonist, which, when provided to a cell or administered to a subject, causes prevention, reduction, alteration, interference, inhibition, or elimination of a NF-κB-related activity.

In an exemplary embodiment, a method for interfering with the activity of nuclear factor-kappaB (NF-κB) may comprise interfering with or reducing NF-κB-mediated suppression of insulin receptor substrate-1 (IRS-1). In an exemplary embodiment, a method for interfering with the activity of nuclear factor-kappaB (NF-κB) may comprise interfering with or reducing NF-κB-mediated suppression of glucose transporter 4 (Glut4). In another exemplary embodiment, a method for interfering with the activity of nuclear factor-kappaB (NF-κB) may comprise interfering with or reducing NF-κB-mediated suppression of adiponectin. In yet another exemplary embodiment, a method for interfering with the activity of nuclear factor-kappaB (NF-κB) may comprise interfering with or reducing NF-κB-mediated expression of monocyte chemoattractant protein-1 (MCP-1).

Another aspect provided herein is a method for decreasing insulin resistance of a cell, comprising: providing to a cell an effective amount of a composition comprising an IGFBP-3 receptor agonist; and decreasing insulin resistance of the cell. Insulin resistance is a condition where a cell is resistant to the effects of insulin. Therefore, the normal cellular response to a given amount of insulin is reduced. As a result of insulin resistance, higher levels of insulin are needed in order for insulin to have its normal effects on a cell.

The term “decreasing insulin resistance of a cell” refers to a difference in degree from a first state, such as an untreated state in a cell, to a second state, such as a treated state in a cell. For example, in the absence of treatment with the methods or compositions of the present invention, insulin resistance may occur at a first rate. If a cell is exposed to treatment with the methods or compositions of the present invention, insulin resistance occurs at a second rate that is altered, decreased, or reduced from the first rate. Thus, the terms “decreasing” “preventing,” “reducing,” “altering,” or “inhibiting” may be used interchangeably through this application and may refer to a partial reduction, substantial reduction, near-complete reduction, complete reduction, or an absence of insulin resistance.

The term “an effective amount” in the context of a method for decreasing insulin resistance of a cell is considered to be any quantity of the IGFBP-3 receptor agonist, which, when provided to a cell or administered to a subject, causes prevention, reduction, alteration, interference, inhibition, or elimination of insulin resistance

Insulin resistance is observed in several cell types, including, but not limited to, adipose cells, muscle cells, and liver cells. Although a decrease in glucose absorption is commonly observed in insulin resistant adipose cells, insulin resistance in adipose cells also causes elevated hydrolysis of stored triglycerides, resulting in elevated levels of free fatty acids in blood plasma. Further, insulin resistance in muscle and liver cells not only results in a decrease in glucose uptake by these cells but also results in impaired glycogen synthesis.

In one embodiment of a method for decreasing insulin resistance of a cell, the method may further comprise increasing uptake of glucose by the cell. For example, increasing the uptake of glucose by a cell can comprise increasing the uptake of glucose by a cell by about at least 50% as compared to a cell not provided with an effective amount of a composition comprising an IGFBP-3 receptor agonist. In some embodiments, increasing the uptake of glucose by a cell can comprise increasing the uptake of glucose by a cell by about at least 100% as compared to a cell not provided with an effective amount of a composition comprising an IGFBP-3 receptor agonist. In some embodiments, increasing the uptake of glucose by a cell can comprise increasing the uptake of glucose by a cell by about at least 200% as compared to a cell not provided with an effective amount of a composition comprising an IGFBP-3 receptor agonist. In some embodiments, increasing the uptake of glucose by a cell can comprise increasing the uptake of glucose by a cell by about at least 300% as compared to a cell not provided with an effective amount of a composition comprising an IGFBP-3 receptor agonist. In some embodiments, increasing the uptake of glucose by a cell can comprise increasing the uptake of glucose by a cell by about at least 400% as compared to a cell not provided with an effective amount of a composition comprising an IGFBP-3 receptor agonist.

Another aspect provided herein is a method for reducing expression of monocyte chemoattractant protein-1 (MCP-1) in a cell, comprising: providing to a cell an effective amount of a composition comprising an IGFBP-3 receptor agonist; and reducing expression of MCP-1 in the cell.

The term “reducing the expression of MCP-1 in the cell” refers to a difference in degree from a first state, such as an untreated state in a cell, to a second state, such as a treated state in a cell. For example, in the absence of treatment with the methods or compositions of the present invention, MCP-1 expression may occur at a first amount. If a cell is exposed to treatment with the methods or compositions of the present invention, MCP-1 expression occurs at a second amount that is altered, decreased, or reduced from the first amount. Thus, the terms “decreasing” “preventing,” “reducing,” “altering,” or “inhibiting” may be used interchangeably through this application and may refer to a partial reduction, substantial reduction, near-complete reduction, complete reduction, or absence of MCP-1 expression. Reducing the expression of MCP-1 in a cell may include interfering with effective action of MCP-1 in cellular pathways in which MCP-1 is active, for example as a signaling factor, or interfering with the transcription of MCP-1 genes or the translation of MCP-1 mRNA, among others.

The term “an effective amount” in the context of a method for reducing the expression of MCP-1 in the cell is considered to be any quantity of the IGFBP-3 receptor agonist, which, when provided to a cell or administered to a subject, causes prevention, reduction, alteration, interference, inhibition, or elimination of MCP-1 expression.

Another aspect proved herein is a method for treating a metabolic syndrome, comprising: administering to a subject having a metabolic syndrome a therapeutically effective amount of a composition comprising an IGFBP-3 receptor agonist. A metabolic syndrome refers to one or more risk factors or symptoms commonly associated with overweight and obese subjects, which increases the risk to the subject of heart disease, diabetes, stroke, and other diseases associated with biochemical processes of the body. For example, metabolic syndrome may comprise one or more symptoms, including, but not limited to, insulin resistance, hyperlipidemias, hypertension, atherosclerosis, any obesity-induced metabolic dysregulation, and diseases attributed to elevated NF-κB activity (e.g., inflammatory disease, Duchenne muscular dystrophy), among others. Although subjects having metabolic syndrome are often obese and overweight, a non-obese or non-overweight subject exhibiting one or more of the above symptoms can be a candidate for the methods and compositions disclosed herein.

The term “treating” as used herein with regards to metabolic syndrome may refer to preventing the condition or disorder, slowing the onset or rate of development of the condition or disorder, reducing the risk of developing the condition or disorder, preventing or delaying the development of at least one symptom associated with the condition or disorder, reducing or ending at least one symptom associated with the condition or disorder, generating a complete or partial regression of the condition or disorder, or some combination thereof.

Embodiments of the methods of treating a metabolic syndrome can comprise administering a therapeutically effective amount of an IGFBP-3 receptor agonist. Administration of an IGFBP-3 receptor agonist may be performed by many known routes of administration, including, but not limited to, topical administration, oral administration, enteral administration, intratumoral administration, parenteral administration (e.g., epifascial, intraarterial, intracapsular, intracardiac, intracutaneous, intradermal, intramuscular, intraorbital, intraosseous, intraperitoneal, intraspinal, intrasternal, intravascular, intravenous, intravesical, parenchymatous, or subcutaneous administration), among others.

The phrase “therapeutically effective amount” as used herein is an amount of a compound that produces a desired therapeutic effect in a subject, such as preventing or treating metabolic syndrome or alleviating one or more symptoms associated with metabolic syndrome. The precise therapeutically effective amount is an amount of the composition that will yield effective results in terms of efficacy of treatment in a given subject. This amount (i.e., dosage) may vary depending upon a number of factors, including, but not limited to, the characteristics of the IGFBP-3 receptor agonist (including activity, pharmacokinetics, pharmacodynamics, and bioavailability), the physiological condition of the subject (including age, sex, disease type and stage, general physical condition, and responsiveness to a given dosage), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly.

A therapeutically effective dose of an IGFBP-3 receptor agonist may be administered daily, more than one time a day, weekly, monthly, or over one or more years to treat or prevent metabolic syndrome and its related symptoms. An effective dose may comprise from about 0.001 μg to about 1,000 mg/kg subject/day of an IGFBP-3 receptor agonist compound. In another embodiment, an effective dose may comprise from about 0.01 μg to about 100 mg/kg subject/day of an IGFBP-3 receptor agonist compound. In yet another embodiment, an effective dose may comprise from about 0.1 μg to about 10 mg/kg subject/day of an IGFBP-3 receptor agonist compound.

Compositions of the present invention may be formulated according to protocols well known in the art. The compositions may be in the form of a powder, tablet, capsule, liquid, ointment, cream, gel, hydrogel, aerosol, spray, micelle, transdermal patch, liposome, or any other suitable form that may be administered to a subject.

In an exemplary embodiment provided herein, a method for treating a metabolic syndrome may comprise administering to a subject having insulin resistance a therapeutically effective amount of a composition comprising an IGFBP-3 receptor agonist. This method may further comprise decreasing insulin resistance of the subject; and increasing the uptake of glucose by the subject. In such methods, increasing the uptake of glucose by a subject may comprise increasing the uptake of glucose by a subject by about at least 50% as compared to a subject not provided with an effective amount of a composition comprising an IGFBP-3 receptor agonist. In some embodiments, increasing the uptake of glucose by a subject may comprise increasing the uptake of glucose by a subject by about at least 100% as compared to a subject not provided with an effective amount of a composition comprising an IGFBP-3 receptor agonist. In some embodiments, increasing the uptake of glucose by a subject may comprise increasing the uptake of glucose by a subject by about at least 200% as compared to a subject not provided with an effective amount of a composition comprising an IGFBP-3 receptor agonist. In some embodiments, increasing the uptake of glucose by a subject may comprise increasing the uptake of glucose by a subject by about at least 300% as compared to a subject not provided with an effective amount of a composition comprising an IGFBP-3 receptor agonist. In some embodiments, increasing the uptake of glucose by a subject may comprise increasing the uptake of glucose by a subject by about at least 400% as compared to a subject not provided with an effective amount of a composition comprising an IGFBP-3 receptor agonist.

In another exemplary embodiment there is provided a method for treating a metabolic syndrome which comprises administering to a subject having atherosclerosis a therapeutically effective amount of a composition comprising an IGFBP-3 receptor agonist. This method may further comprise reducing the expression of MCP-1 in the subject.

The methods of treating disclosed in the present invention are not limited to methods of treating metabolic syndrome. The methods of the present invention can be used to treat many diseases associated with uncontrolled NF-κB activity, including, but not limited to, various cancers, obstructive respiratory disorders such as severe corticosteroid-dependent asthma, rheumatoid arthritis, juvenile arthritis, Crohn's Disease, psoriasis, sarcoidosis, Duchenne muscular dystrophy, and Behcet's disease.

The IGF system is a multicomponent network of molecules that is ubiquitously involved in the regulation of growth, proliferation, and differentiation of a variety of cell types. For example, IGF-I appears to be involved in the inflammatory process associated with bronchial asthma. IGF-1 activity is modulated by IGFBPs. Although the six IGFBPs display high levels of conservation in their C- and N-terminal domains, their expression patterns and properties vary widely. Of the six IGFBPs, IGFBP-3 is the most abundant in serum. IGFBP-3 is known to modulate IGF-1 activity in certain situations, but its pathophysiological role in respiratory inflammation and hyperresponsiveness has not been described.

In the studies presented herein, a mouse model for asthma was used to determine the effects of the wild type-IGFBP-3 and mutant IGFBP-3GGG on respiratory inflammation and airway hyperresponsiveness. Western blot analysis of lung tissue and enzyme immunoassays of broncheolar lavage fluid revealed that expression of IL-4, IL-5, IL-13, TNF-α, IL-1β, VCAM-1, ICAM-1, eotaxin, and RANTES increased following challenge with OVA, and that this increase was greatly reduced by administration of WT-AdIGFBP-3 or m-AdIGFBP-3GGG. Western blot analysis also confirmed that endogenous IGFBP-3 levels were significantly reduced following challenge with OVA, while endogenous IGF-1 levels were significantly increased.

The results provided herein demonstrate that IGFBP-3 is a potent inhibitor of the respiratory inflammation and airway hyperresponsiveness associated with obstructive respiratory disorders such as bronchial asthma. The IGFBP-3 mutant IGFBP-3GGG, which lacks the ability to bind IGF, has nearly the same inhibitory effectiveness as the wild-type protein, demonstrating that inhibition is the result of intrinsic IGFBP-3 anti-inflammatory activity rather than merely the ability to block IGF activity. The present results indicate that alterations in IGFBP-3 levels are implicated in the pathogenesis of bronchial asthma and other obstructive respiratory disorders, and that restoration of IGFBP-3 may serve to prevent and suppress these disorders. These results are consistent with clinical studies which have suggested a role for insufficient IGFBP-3 in the pathogenesis of inflammatory diseases, for example demonstrating that serum IGFBP-3 was significantly decreased in subjects with a variety of inflammatory diseases including juvenile idiopathic arthritis, rheumatoid arthritis, pulmonary sarcoidosis, cystic fibrosis, Crohn's disease and inflammatory bowel disease and when disease was in remission the serum IGFBP-3 reached normal levels.

Without wishing to be bound by any proposed mechanism of action, it is suggested that IGFBP-3 degrades IκBα and p65-NF-κB proteins through IGFBP-3 receptor, thereby inhibiting TNF-α-induced activation of NF-κB signaling cascades, and consequently the IGFBP-3/IGFBP-3R system may play a role in the pathogenesis of asthma and may serve as a potential therapeutic target for this and other obstructive respiratory disorders.

The results provided herein demonstrate that IGFBP-3 receptor agonistic antibodies have potential for treatment of obstructive respiratory disorders and autoimmune diseases.

A mouse model for asthma was used to determine the effects of IGFBP-3 on respiratory inflammation and airway hyperresponsiveness. Three adenoviral vectors were generated for these studies. The first, WT-AdlGFBP-3, contained cDNA encoding wild-type IGFBP-3. The second, m-AdlGFBP-3, contained cDNA encoding the GGG-IGFBP-3 mutant. The third, AdLacZ, was used as a control. The results provided herein demonstrate that IGFBP-3 and the IGFBP-3 mutant IGFBP-3GGG, which lacks the ability to bind IGF are potent inhibitors of the respiratory inflammation and airway hyperresponsiveness associated with obstructive respiratory disorders such as bronchial asthma in a mouse model. The present results indicate that the effect of IGFBP-3 on respiratory inflammation and airway hyperresponsiveness is mediated through IGFBP-3R. Thus, embodiments of the present invention are directed to methods and compositions for the treatment of asthma and other respiratory inflammation diseases. In particular, the invention relates to compositions comprising IGFBP-3 receptor agonist antibodies and methods for the treatment of asthma and other respiratory inflammation diseases with IGFBP-3 receptor agonist antibodies.

Embodiments of the methods of treating obstructive respiratory disorders such as asthma and other respiratory inflammation diseases may comprise administering a therapeutically effective amount of an IGFBP-3 receptor agonist. In one embodiment the method may comprise the systemic administration of a therapeutically effective amount of an IGFBP-3 receptor agonist. Administration of an IGFBP-3 receptor agonist may be performed by many known routes of administration, including, but not limited to, topical administration, oral administration, enteral administration, intratumoral administration, parenteral administration (e.g., epifascial, intraarterial, intracapsular, intracardiac, intracutaneous, intradermal, intramuscular, intraorbital, intraosseous, intraperitoneal, intraspinal, intrasternal, intravascular, intravenous, intravesical, parenchymatous, or subcutaneous administration), among others.

As used herein in relation to the treatment of an obstructive respiratory disorder the phrase “therapeutically effective amount” is an amount of a compound that produces a desired therapeutic effect in a subject, such as the alleviation of asthma or other respiratory inflammation diseases. The precise therapeutically effective amount is an amount of the composition that will yield effective results in terms of efficacy of treatment in a given subject. This amount (i.e., dosage) may vary depending upon a number of factors, including, but not limited to, the characteristics of the IGFBP-3 receptor agonist (including activity, pharmacokinetics, pharmacodynamics, and bioavailability), the physiological condition of the subject (including age, sex, disease type and stage, general physical condition, and responsiveness to a given dosage), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly.

A therapeutically effective dose of an IGFBP-3 receptor agonist may be administered daily, more than one time a day, weekly, monthly, or over one or more years to treat or prevent metabolic syndrome and its related symptoms. An effective dose may comprise from about 0.001 μg to about 1,000 mg/kg subject/day of an IGFBP-3 receptor agonist compound. In another embodiment, an effective dose may comprise from about 0.01 μg to about 100 mg/kg subject/day of an IGFBP-3 receptor agonist compound. In yet another embodiment, an effective dose may comprise from about 0.1 μg to about 10 mg/kg subject/day of an IGFBP-3 receptor agonist compound. For example, the subject may be injected with the antibody at regular intervals such as by injection of the antibody twice per week at 100 μg per injection.

The results provided herein demonstrate that IGFBP-3 and IGFBP-3 receptor agonistic antibodies are potent inhibitors of respiratory inflammation and airway hyperresponsiveness associated with obstructive respiratory disorders such as bronchial asthma. The IGFBP-3 mutant GGG-IGFBP-3 showed nearly the same inhibitory effectiveness as the wild-type protein, demonstrating that inhibition is the result of intrinsic IGFBP-3 anti-inflammatory activity rather than merely the ability to block IGF activity. The results described herein indicate that alterations in IGFBP-3 levels are implicated in the pathogenesis of bronchial asthma and other obstructive respiratory disorders, and that restoration of IGFBP-3 will serve to prevent and suppress these disorders.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

It must be noted that, as used in this specification and the appended claims, the singular forms “a,” “an,” and “the” include plural referents unless the context clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All patents, patent applications and references included herein are specifically incorporated by reference in their entireties.

Throughout this description, various components may be identified as having specific values or parameters, however, these items are provided as exemplary embodiments. Indeed, the exemplary embodiments do not limit the various aspects and concepts of the present invention as many comparable parameters, sizes, ranges, and/or values may be implemented. It should be understood, of course, that the foregoing relates only to exemplary embodiments of the present invention and that numerous modifications or alterations may suggest themselves to those skilled in the art without departing from the spirit and the scope of the invention as set forth in this disclosure.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

### EXAMPLES

### Example 1

### Effect of IGFBP-3 on TNF-α-Induced Insulin Resistance

Materials and Methods.

Cell culture was performed using the following materials: DMEM (low glucose, Invitrogen Cat#11885084), FBS (VWR Cat#MTT35011CV), isobutyl-methylanthine (Sigma 17018), dexamethasone (Sigma D4902), indomethacin (Sigma 17378), insulin (Sigma 19278), and TNF-α (Sigma T6674). The antibodies of insulin receptor substrate-1 (IRS-1) (sc-559), GLUT4 (sc-1608), MCP-1 (sc-32786), insulin receptor β subunit (sc-711) were purchased from Santa Cruz Biotechnology (Santa Cruz, Calif.). The mouse anti-adiponectin human monoclonal antibody (MAB3604) was from CHEMICON International, Inc. The monoclonal anti-α-tubulin antibody (T9026) was from Sigma-Aldrich, Inc. Anti-mouse IgG antibody conjugated to horseradish peroxidase (#7076), and anti-rabbit IgG antibody conjugated to horseradish peroxidase (#7074) were from Cell Signal Technology.

Cell Differentiation.

Preadipocyte cells were purchased from Lonza Biologics Inc. and were plate at a density of 2 to 5×10³/cm² and grow at 37° C. in an atmosphere of 95% air and 5% CO₂. Cells were grown to 100% confluence in the growth medium supplemented with 10% FBS. Three-day post-confluent cells were incubated in adipogenesis-inducing medium (AM) (DMEM, 1.0 g/L glucose, 0.5 mM isobutyl-methylanthine, 1 μM dexamethasone, 0.2 mM indomethacin, 10 μM insulin, 10% FBS) for 6 days (the medium was changed every 3 days), incubated 1 day in adipogenesis maintenance medium (MM) (DMEM, 1.0 g/L glucose, 10 μM insulin, 10% FBS) and then switched to AM again.

Oil Red O Staining.

Cells were stained with Oil Red O as described previously. Briefly, cells were fixed in 10% solution of formaldehyde in aqueous phosphate buffer for 1 hour or more, washed with 60% isopropanol, and stained with Oil Red O solution (in 60% isopropanol) for 10 minutes followed by repeated washing with water, and destained in 100% isopropanol for 15 minutes. The optical density (OD) of the solution was measured at 500 nm.

RT-PCR Analysis.

Total RNA was isolated using Trizol (Life Technologies #15596-026) according to the manufacturer's protocol. To analyze gene express by PCR, 0.5 μg of total RNA was primed with ThermoScript RT (Invitrogen #11146-016) in 20 μl of volume, and 2 μl of the total volume was used for subsequent PCR experiments. Primers and PCR conditions are as follows: IRS-1 (213 bp) forward primer 5′-CCTGGATTTGGTCAAGGACT-3′, reverse primer 5′-TCATTCTGCTGTGATGTCCA-3′; Glut4 (192 bp) forward primer 5′-TTATTCGACCAGCATCTTCG-3′, reverse primer 5′-AGCAGAGCCACAGTCATCAG-3′; adiponectin (214 bp) forward primer 5′-TGCTGGGAGCTGTTCTACTG-3′, reverse primer 5′-GTTTCACCGATGTCTCCCTT-3′; MCP-1 forward primer 5′-ATCAATGCCCCAGTCACC-3′, reverse primer 5′-AGTCTTCGGCGTTTGGG-3′. IRS-1 and adiponectin PCRs were performed using PCR buffer (Invitrogen) supplemented with 1.5 mM MgCl₂ at 94° C. for 45 s, 56° C. for 45 s, 72° C. for 60 s for 27 cycles. Glut-4 PCR was performed at 94° C. for 45 s, 54° C. for 45 s, 72° C. for 60 s for 30 cycles. MCP-1 and 1432M PCRs performed at 94° C. for 45 s, 55° C. for 45 s, 72° C. for 60 s for 25 cycles. Reaction products were resolved on 2% agarose gels.

Effect of IGFBP-3 on TNF-α-Induced Insulin Resistance.

Insulin resistance is a major risk factor for type II diabetes as well as hypertension, dyslipidemia, and atherosclerosis (Reaven, 1988). Despite several investigations, the molecular mechanism underlying insulin resistance has not been adequately elucidated. TNF-α is an adipocytokine and induces insulin resistance (Hotamisligil, 1993). A TNF-α signal results in the phosphorylation of Ser307 of insulin receptor (IR) substrate 1 (IRS-1), in turn attenuating the metabolic insulin signal (Kanety et al., 1995). Many serine kinases, such as JNK, glycogen synthase kinase 3, and the mammalian target of rapamycin, have been reported to phosphorylate serine residues of IRS-1 (Gao et al., 2002). A recent study showed the IKK complex phosphorylated IRS-1 at Ser307, which is associated with TNF-α stimulation and diminished insulin signaling (Gao et al., 2002).

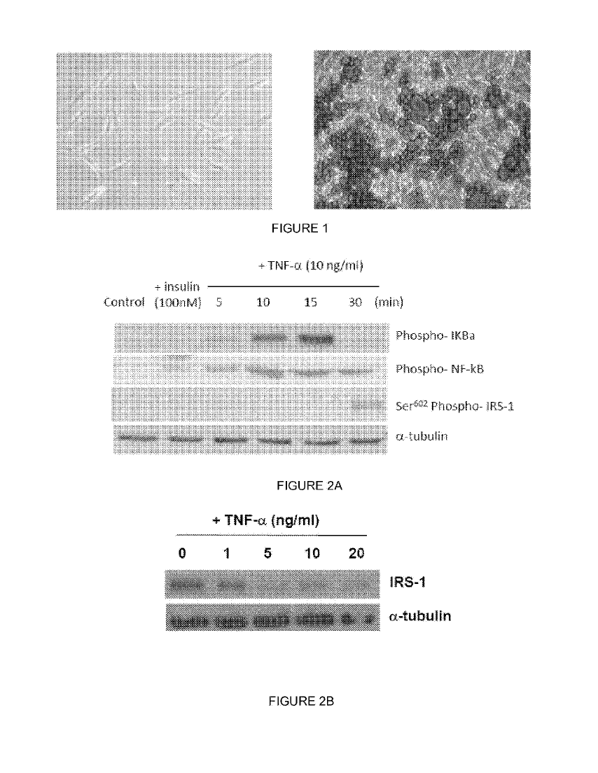

Using an in vitro system, the effect of IGFBP-3 on TNF-α-induced insulin resistance was investigated. As shown in FIG. 1, human preadipocytes were fully differentiated to adipocytes within 14 days as demonstrated Oil Red staining when cells were cultured in adipogenesis-inducing medium.

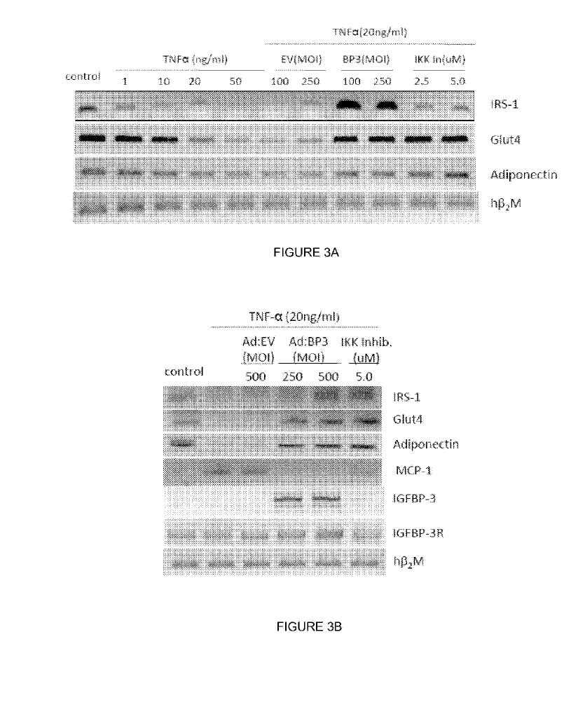

Fully differentiated human adipocytes were treated with TNF-α to mimic chronic inflammatory condition seen in patients with obesity. Treatment of cells with 10 ng/ml of TNF-α resulted in activation of NF-κB activity, as shown by induction of phosphorylation of IκBα and p65-NF-κB proteins in a time dependent manner (FIG. 2A). Concomitantly, TNF-α induced serine phosphorylation of IRS-1 protein at position 602, which indicates inhibition of IRS-1 function, similar to phosphorylated IRS-1 at Ser307, thereby attenuating the metabolic insulin signal. Furthermore, treatment with TNF-α resulted in suppression of IRS-1 production, which further inhibits insulin signal (FIG. 2B).

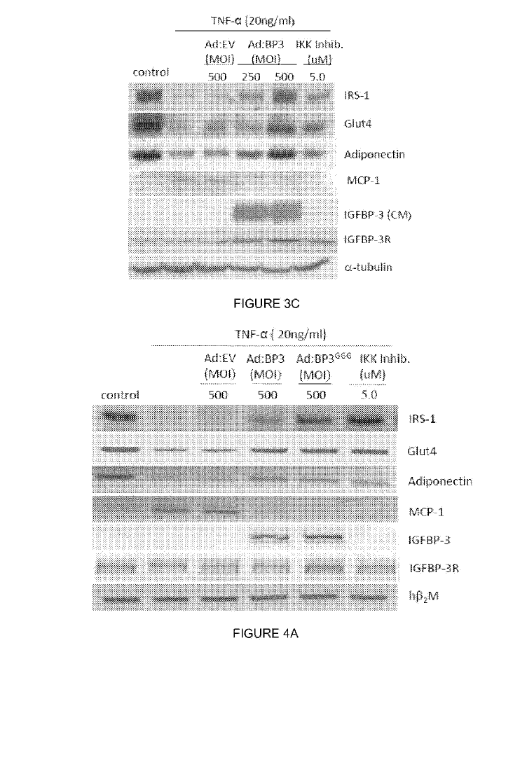

In order to investigate the effect of IGFBP-3 on TNF-α-induced pro-inflammatory condition in human adipocytes, cells were infected with adenovirus, containing IGFBP-3 cDNA (Ad:IGFBP-3), in the presence of TNF-α. TNF-α treatment resulted in suppression of IRS-1, glucose transporter 4 (Glut 4), and adiponectin at the mRNA level (FIGS. 3A and 3B) and the protein level (FIG. 3C). TNF-α-induced suppression of IRS-1, Glut 4, and adiponectin was attenuated by co-treatment with the IKK inhibitor. The IKK inhibitor inhibits NF-κB signal by blocking phosphorylation of IκBα. Similarly, infection with adenovirus expressing IGFBP-3 (Ad:IGFBP-3) but not control empty adenovirus vector (Ad:EV) shows suppression of those proteins. These results indicate that IGFBP-3 inhibits TNF-α-induced activation of NF-κB signaling. As shown in FIGS. 3B and 3C, expression of IGFBP-3 receptor (IGFBP-3R) was not significantly changed after TNF-α or IKK inhibitor treatment. On the other hand, TNF-α-induced MCP-1 was completely inhibited by infection with Ad:IGFBP-3 at both the mRNA level (FIG. 33B) and the protein level (FIG. 3C).

To elucidate whether the mechanism involves IGF-independent actions of IGFBP-3, studies were also conducted using a mutant-IGFBP-3, IGFBP-3^{GGG}, which has a reduced binding capacity for IGFs as compared to wild-type IGFBP-3. TNF-α-induced suppression of IRS-1, Glut 4, and adiponectin was significantly reduced by the administration of an adenovirus expressing the IGFBP-3^{GGG} mutant (Ad:IGFBP-3^{GGG}) (FIGS. 4A and 4B). In addition, TNF-α-induced increase of MCP-1 was also inhibited by treatment with mutant-AdIGFBP-3^{GGG} similar to wild type IGFBP-3. These findings suggest that inhibitory effect of IGFBP-3 on the biological function of TNF-α in human adipocytes is independent of IGFs.

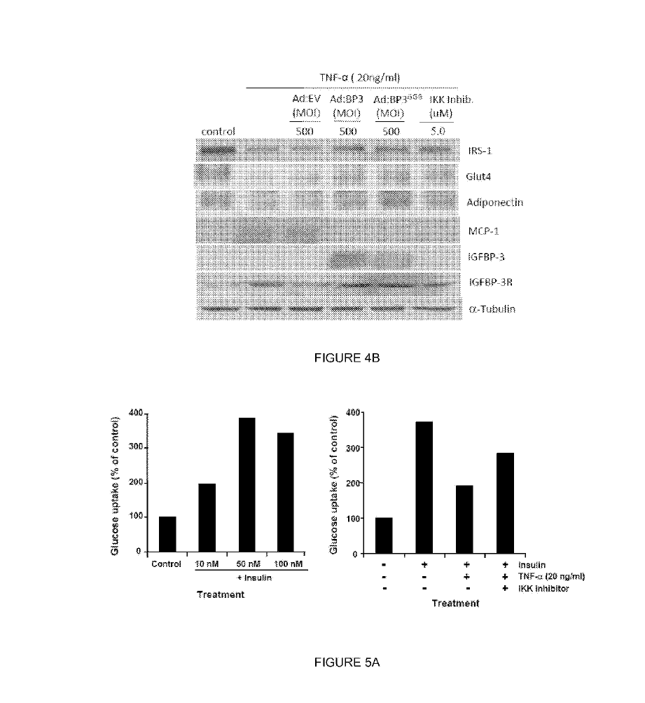

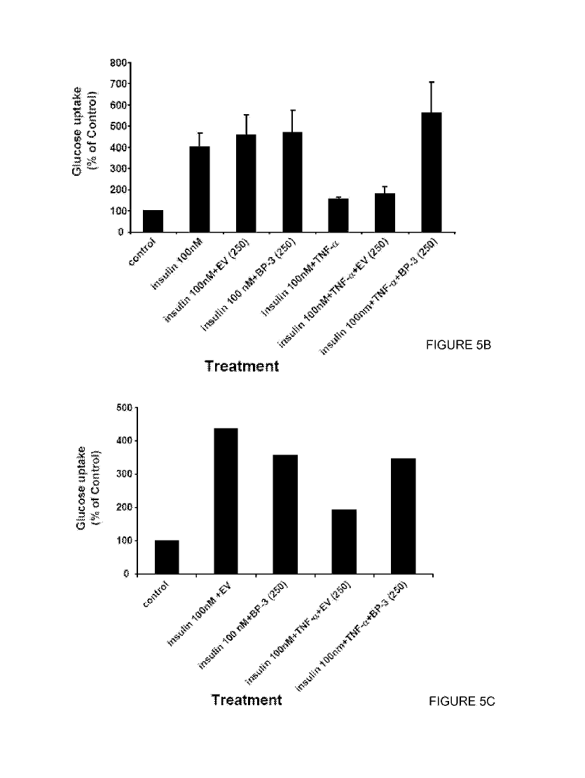

In order to characterize the effect of IGFBP-3 on TNF-α-induced insulin resistance, [³H]glucose uptake assays were employed. As demonstrated in FIG. 5A, insulin glucose uptake occurred in a concentration dependent manner, whereas co-treatment with TNF-α resulted in inhibition of insulin-induced glucose uptake in human adipocytes. Furthermore, the IKK inhibitor attenuates TNF-α-induced suppression of glucose uptake. Similarly, IGFBP-3 treatment in the presence of insulin and TNF-α resulted in attenuation of TNF-α-induced suppression of glucose uptake (FIG. 5B). IGFBP-3 treatment shows no effect on insulin-induced increase of glucose uptake in the absence of TNF-α. These data clearly indicate that IGFBP-3 interferes with TNF-α-induced NF-κB signaling thereby affecting the inhibitory effect of TNF-α on insulin-induced glucose uptake without affecting insulin action. These IGFBP-3 effects on TNF-α-induced insulin resistance were also observed in mouse 3T3 adipocytes (FIG. 5C).

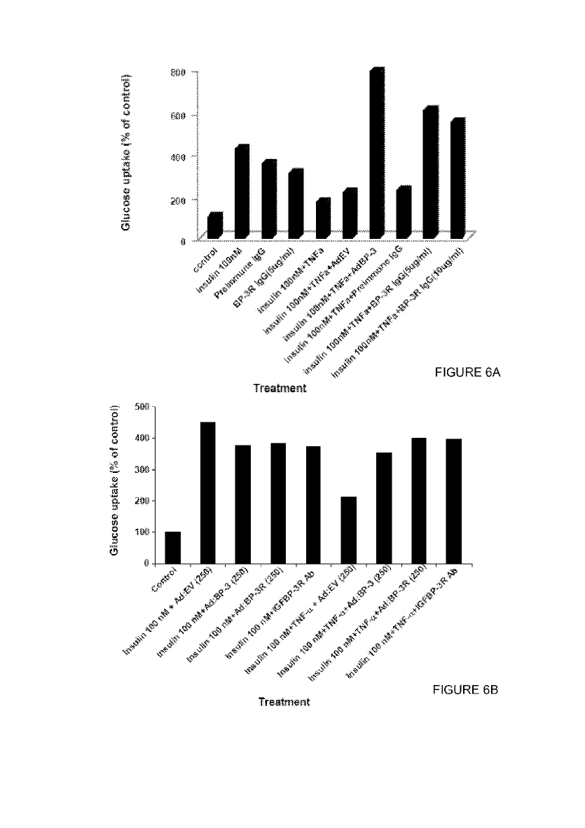

Current IGFBP-3 and mutant-IGFBP-3^{GGG} sensitizing effects on TNF-α-induced insulin resistance strongly suggest that IGFBP-3 receptor is involved in these biological processes. Thus, IGFBP-3 receptor agonistic antibodies should mimic the biological effect observed with IGFBP-3 treatment. To test this hypothesis, adipocytes were treated with purified IgG IGFBP-3 receptor antibodies or preimmune sera in the presence of insulin and TNF-α. As shown in FIG. 6, IGFBP-3 receptor antibodies, but not preimmune sera, restored TNF-α-induced inhibition of glucose uptake in human adipocytes (FIG. 6A), as well as mouse 3T3 adipocytes (FIG. 6B). Taken together, these results support the conclusion that IGFBP-3 (or other IGFBP-3 receptor agonists) can inhibit TNF-α-induced insulin resistance by inhibiting TNF-α-induced NF-κB activity in adipocytes. Therefore, IGFBP-3 and other IGFBP-3 receptor agonists have therapeutic potential for type II diabetes as well as hypertension, dyslipidemia, and atherosclerosis.

### Example 2

### Effect of IGFBP-3 on MCP-1 Expression

MCP-1 is a member of the CC chemokine family and promotes migration of inflammatory cells by chemotaxis and integrin activation, and it has been reported to recruit monocytes from the blood into atherosclerotic lesions, thereby promoting foam cell formation (Boring 1998, Gu 1998, Linton 2003). MCP-1 in adipose tissue and plasma MCP-1 levels have been found to positively correlate with the degree of obesity (Weisberg 2003, Xu 2003, Christiansen 2005, and Sartipy 2003). In addition, increased expression of this chemokine in adipose tissue precedes the expression of other macrophage markers during the development of obesity. (Xu 2003). A recent report on mice lacking C—C motif chemokine receptor-2 (CCR2), a receptor for MCP-1 and several other chemokines, suggested the MCP-1/CCR2 pathway influences the development of obesity and insulin resistance via adipose macrophage accumulation and inflammation. (Weisberg 2006). Beyond glucose lowering, thiazolidinediones (TZDs), agonists of the peroxisome proliferator-activated receptor (PPAR)γ, improve various factors associated with cardiovascular risk; however, whether the effects of TZDs translate into beneficial cardiovascular outcomes remains controversial (Khanderia 2009). Although the first large-scale clinical trial evaluating the effect of a TZD on secondary prevention of major adverse cardiovascular outcomes supported this hypothesis, a recently published meta-analysis raised substantial uncertainty about the cardiovascular safety of rosiglitazone, a TZD. In addition, TZDs exert a broad array of pleiotropic effects. For example, TZD-related fluid retention can exacerbate or lead to heart failure. Several meta-analyses also associate rosiglitazone with an increased risk of myocardial ischemic events.

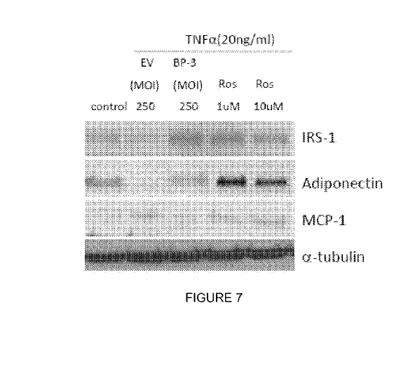

In order to investigate potential different biological effects between IGFBP-3 and TZDs in cardiovascular disease, adipocytes were treated with 20 ng/ml of TNF-α followed by treatment with IGFBP-3 or rosiglitazone (Ros). Rosiglitazone treatment resulted in attenuation of TNF-α-induced suppression of IRS-1 and adiponectin, similar to the data with IGFBP-3 treatment (FIG. 7). However, rosiglitazone, unlike IGFBP-3, was unable to suppress TNF-α-induced increase of MCP-1. Since MCP-1 is a key player to recruit monocytes from the blood into atherosclerotic lesions, thereby promoting foam cell formation, IGFBP-3 not only sensitizes insulin resistance but also may prevent the incidence of cardiovascular disease, as atherosclerosis may be caused by elevated MCP-1 production in adipocytes.

### Example 3

### IGFBP-3 and Adipose Tissue-Mesechymal Stem Cells

Adipose tissue, like bone marrow, is a mesodermally derived tissue, which contains stem cells. Adipose tissue-mesenchymal stem cells (AT-MSC) share many of the characteristics of their bone marrow counterpart, including intrinsic preferential migratory ability toward tumors, including breast tumors, extensive proliferation potential, and the ability to undergo multi-lineage differentiation (Kern 2006, Strem 2005, Wanger 2005, Kucerova 2008). The yield of MSC from adipose tissue is about 40-fold higher compared with the bone marrow (Kern 2006). Adipose tissue contains not only adipogenic progenitor cells, but also multipotent stem cells, which can differentiate into fat, bone, cartilage, and other types of tissue (Zuk 2001). Recent use of the AT-MSC-rich lipotransfer for cosmetic breast augmentation indicates that local delivery of AT-MSC is safe and effective with regards to its proliferation and differentiation into adipocytes, strongly suggesting potential use of AT-MSC for a local cell-based delivery of cyto-reagents to breast tissue (Yoshimura 2008). Furthermore, the source of autologous stem cells for personalized cell-based therapy is of minimal risk to the donor and possesses no ethical concerns. It suggests that AT-MSC may be a promising source of autologous stem cells in personalized cell-based therapies for human disease. Therefore, autologous injection of genetically engineered AT-MSC that overexpress IGFBP-3 has therapeutic potential for treatment of above mentioned diseases.

### Example 4

### Generation of Rabbit Polyclonal IGFBP-3 Receptor (IGFBP-3R) Agonistic Antibodies

Rabbit polyclonal antibodies were generated against GST-fused IGFBP-3R [915 base pairs encoding a 240 amino acid polypeptide (GenBank accession #FJ748884)1. GST::IGFBP-3R protein was generated by subcloning the IGFBP-3R cDNA into the pGEX-4T-1 vector in frame with GST, transforming into E. coli strain BL21(DE3)pLysS, and inducing expression with IPTG. Gel-purified protein from cell lysates was injected into rabbits for polyclonal IGFBP-3R antibody production. The IGFBP-3R agonistic antibody was further purified using purification Affi-Gel Protein A MAPS II Kits. Briefly, 2 ml of Affi-Gel was activated with 10 ml of sample binding buffer in a column, and the 3 ml of IGFBP-3R antisera diluted with the same volume of sample binding buffer was applied to the column. The column was washed with 30 ml of binding buffer. The elution was performed twice, first with 10 ml of elution buffer, and then with 20 ml of elution buffer. The eluate was collected in 1 ml fractions, and 107 μl of 1M Tris (pH 9.0) was added to each ml of the eluate. The each fraction was measured for protein concentration using Pierce BCA Protein Assay Kit.

### Example 5

### Anti-Inflammatory Effect of IGFBP-3R Agonistic Antibodies

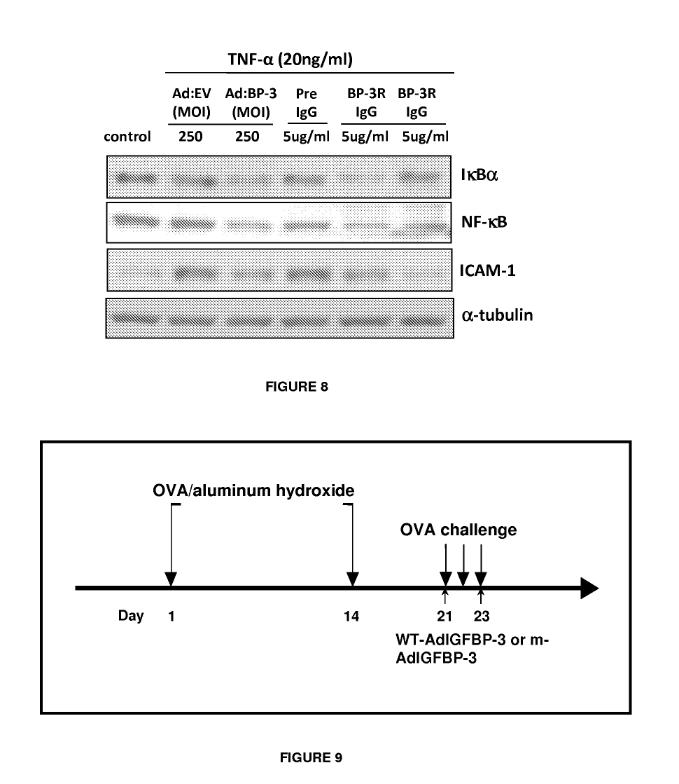

IGFBP-3 and IGFBP-3 receptor agonistic antibodies inhibit TNF-α-induced NF-κB activity and subsequent inflammatory response in BEAS-2B human normal lung epithelial cells. TNF-α (20 ng/ml) treatment was conducted to induce an inflammatory response. BEAS-2B cells were treated with TNF-α for 24 hrs. IGFBP-3 treatment resulted in a decrease of total IκBα and p65-NF-κB levels, thereby blocking TNF-α-induced ICAM-1 expression. Intriguingly, the purified IGFBP-3R antibody shows the same biological effect as seen with IGFBP-3 treatment. As shown in the FIG. 8, treatment with 5 ug/ml of the purified IGFBP-3R agonistic antibody, but not preimmune IgG resulted in a complete suppression of TNF-α-induced ICAM-1 expression as well as a decrease of IκBα and p65-NF-κB expression. These findings illustrate that IGFBP-3R agonistic antibodies have therapeutic potential for treatment of TNF-α-induced inflammatory diseases including obstructive respiratory disorders.

### Example 6

### Characterization of a Murine Asthma Model

Three adenoviral vectors were generated for these studies. The first, WT-AdlGFBP-3, contained cDNA encoding wild-type IGFBP-3. The second, m-AdlGFBP-3, contained cDNA encoding the GGG-IGFBP-3 mutant. The third, AdLacZ, was used as a control.

In the mouse model, mice were sensitized by intraperitoneal injection of OVA. Mice were sensitized on days 1 and 14 by intraperitoneal injection of 20 μg ovalbumin (OVA)(Sigma-Aldrich, St. Louis, Mo., USA) emulsified in 1 mg of aluminum hydroxide (Pierce Chemical Co., Rockford, Ill., USA) in a total volume of 200 μl. Following the initial sensitization, mice were challenged on days 21, 22, and 23 with an aerosol of 3% (wt/vol) OVA in saline using an ultrasonic nebulizer (NE-U12; Omron Corp., Tokyo, Japan) for 30 minutes per day. Control mice received saline in place of OVA.

The adenoviral vectors were administered to the mice intratracheally 21 days after the initial sensitization. Ad vectors (10⁹ plaque-forming units) were administered intratracheally on day 21 (one hour prior to airway challenge with OVA) and day 23 (three hours after airway challenge). Control mice were administered with saline. A schematic of the administration protocol is shown in FIG. 9. This protocol resulted in five experimental groups: SAL+SAL, OVA+SAL, OVA+AdWT-IGFBP-3, OVA+m-AdlGFBP-3, and OVA+AdLacZ.

BAL was performed and the lungs were removed for analysis. Bronchoalveolar lavage (BAL) was performed 72 hours after the last airway challenge on six mice from each experimental group. At the time of lavage, the mice were sacrificed with an overdose of sodium pentobarbitone (pentobarbital sodium, 100 mg/kg body weight, administered intraperitoneally). The chest cavity was exposed to allow for expansion, after which the trachea was carefully intubated and the catheter secured with ligatures. Prewarmed 0.9% NaCl solution was slowly infused into the lungs and withdrawn. BAL aliquots were pooled and stored at 4° C. Part of each pool was then centrifuged and the supernatants were stored at −70° C. until use.

Total cell numbers were counted with a hemocytometer. Smears of BAL cells were prepared by cytospin (Shandon Scientific Ltd., Cheshire, United Kingdom). The smears were stained with Diff-Quik solution (Dade Diagnostics of Puerto Rico Inc., Aguada, Puerto Rico) in order to examine the cell differentials. Two independent, blinded investigators counted the cells using a microscope. Approximately 400 cells were counted in each of four different random locations. The variation in results between the investigators was less than 5%. The mean of the values from the two investigators was used for each cell count. The number of total cells, eosinophils, lymphocytes, and neutrophils in BAL fluid was significantly increased at 72 hours after challenge with OVA (FIG. 2, compare “SAL+SAL” and “OVA+SAL”). The number of each cell type in OVA-challenged BAL fluid was significantly reduced by administration of WT-AdlGFBP-3 and m-AdlGFBP-3

BAL fluid from mice administered with WT-AdlGFBP-3 or m-AdlGFBP-3 displayed significantly reduced numbers of eosinophils, lymphocytes, neutrophils, and total cells. Similar results were obtained when mice were administered with recombinant IGFBP-3. Increased numbers of eosinophils are believed to be associated with many of the tissue changes seen in asthmatic airways, including epithelial damage, thickening of the basement membrane, and the release of mediators with the capacity to cause bronchial smooth muscle contraction and exudation of plasma, resulting in thickening of the airway wall.

Histological studies of the excised lung tissue revealed that mice treated with WT-AdIGFBP-3 or m-AdlGFBP-3 showed markedly reduced levels of inflammation and inflammatory cell infiltration in both the peribronchiolar and perivascular regions. The histological data also confirmed that mice administered with the adenoviral vectors displayed increased expression of IGFBP-3, confirming the effectiveness of expression from the adenoviral vectors. Western blot analysis of lung tissue and enzyme immunoassays of BAL fluid revealed that expression of IL-4, IL-5, IL-13, TNF-α, IL-1β, VCAM-1, ICAM-1, eotaxin, and RANTES increased following challenge with OVA, and that this increase was greatly reduced by administration of WT-AdlGFBP-3 or m-AdlGFBP-3. Western blot analysis also confirmed that endogenous IGFBP-3 levels were significantly reduced following challenge with OVA, while endogenous IGF-1 levels were significantly increased.

Various breathing parameters were measured in response to increasing methacholine concentrations in live, unrestrained mice. The parameters were used to generate a Penh value, and the increase in baseline Penh was used to assess airway responsiveness to methacholine. OVA-challenged mice exhibited airway hyperresponsiveness compared to control mice, as demonstrated by higher Penh values at each methacholine concentration tested. Administration of WT-AdlGFBP-3 or m-AdlGFBP-3 to OVA-challenged mice resulted in a significant decrease in airway hyperresponsiveness, which was indicated by a substantial decrease in Penh values. Similar results were obtained when mice were administered with recombinant IGFBP-3.

### Example 7

### Treatment of Cancer Cells with IGFBP-3R Agonist Antibodies

Cancers are generally classified by the type of cell that the cancer cell resembles. These types include carcinoma, sarcoma, lymphoma and leukemia, germ cell tumor and blastoma. Carcinoma is a cancer derived from epithelial cells and includes the most common types of cancer including breast, prostate, lung and colon cancers; sarcoma is a cancer derived from connective tissue, or mesenchymal cells; lymphoma and leukemia are cancers derived from hematopoietic (blood-forming) cells; germ cell tumor is a cancer derived from pluripotent cells; and blastoma is a cancer derived from immature “precursor” or embryonic tissue.

Cancer, or malignant neoplasm, is a class of diseases in which the cancer cells display uncontrolled growth, invasion that may intrude upon and destroy adjacent tissues, and sometimes metastasize, i.e. spread to other locations in the body via the lymphatic system or by blood. These three malignant properties of cancer cells differentiate them from benign tumors, which do not invade or metastasize.

Embodiments of the invention include methods for treating a cancer. The cancer may be any type of cancer. The methods for treating cancer may comprise administering to a subject having or suspecting of having cancer a therapeutically effective amount of a composition comprising an IGFBP-3 receptor agonist. The IGFBP-3 receptor agonist may comprise many compounds including, but not limited to, IGFBP-3, a portion of IGFBP-3, a receptor agonist antibody or a fragment thereof capable of binding at least a portion of an IGFBP-3 receptor, or a vector capable of expressing at least a portion of IGFBP-3. In one embodiment, the vector comprises an adenovirus expressing at least a portion of IGFBP-3. In some embodiments, administering to a subject a therapeutically effective amount of a composition comprises administering from about 0.001 μg to about 1,000 mg/kg subject/day of the composition.

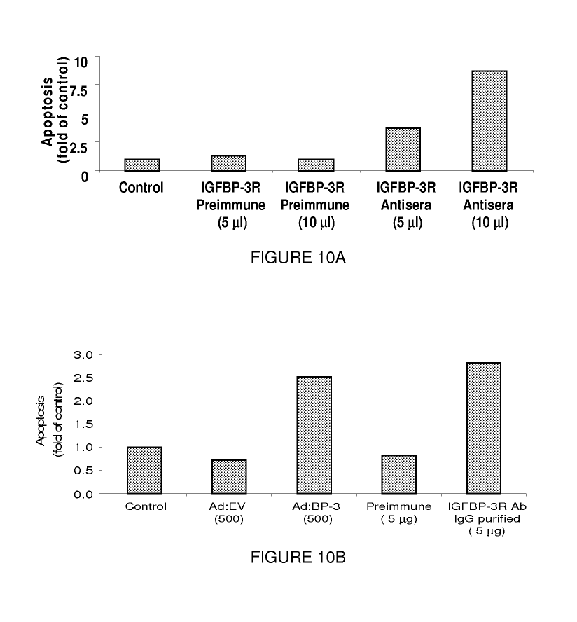

The IGFBP-3 receptor agonists have demonstrated the ability to induce apoptosis in certain cancer cells without an adverse effect on healthy cells. Studies using IGFBP-3R agonist antibodies clearly demonstrate that treatment of IGFBP-3R agonistic antibodies, but not preimmune sera, resulted in induction of apoptosis in human prostate cancer cells. As shown in FIG. 10A, the potency of the IGFBP-3 receptor agonistic antibodies for induction of apoptosis was comparable with that of IGFBP-3.

Recent studies demonstrated that IGFBP-3 shows no induction of apoptosis in human non-malignant cells, whereas IGFBP-3 treatment induced apoptosis in human malignant cells (Lee Y C, Jogie-Brahim S, Lee D Y; Han J; Harada A; Murphy L J; Oh Y. IGFBP-3 blocks the effects of asthma by negatively regulating NF-κB signaling through IGFBP-3R-mediated activation of caspases J. Biol. Chem, 2011, 286(20):17898-17909 hereby incorporated by reference). Taken together, these findings demonstrate the preferential antitumor effect of IGFBP-3 in cancer and therapeutic efficacy of IGFBP-3R agonist antibodies.

The experimental data demonstrates the efficacy of agonistic IGFBP-3R antibodies for induction of apoptosis in cancer cells. As shown in FIG. 10B, the treatment of IgG purified IGFBP-3R antibodies, but not preimmune sera, resulted in induction of apoptosis in human prostate cancer cells. This data is complemented by the demonstrated potency of these agonistic antibodies for induction of apoptosis that is comparable to the potency of IGFBP-3. In the study, IgG purified antibodies were administered two times per day for 3 days. The apoptotic cell death was measured by amounts of cleaved PARP (Poly (ADP-ribose) polymerase).

### REFERENCES

Allen J. T., Bloor C. A., Knight R. A., Spiteri M. A. Am. J. Respir. Cell Mol. Biol. 19, 250-258, 1998.
Blackman M. R., Muniyappa R., Wilson M., Moquin B. E., Baldwin H. L., Wong K. A., Snyder C., Magalnick M., Alli S., Reynolds J., Steinberg S. M., Goldbach-Mansky R. Arthritis Res. Ther. 9, R73, 2007.
Boring, L., Gosling, J., Cleary, M., and Charo, I. F. Nature 394, 894-897, 1998.
Christiansen, T., Richelsen, B., and Bruun, J. M. Int. J. Obes. Relat. Metab. Disord. 29, 146-150, 2005.
De Benedetti F., Meazza C., Oliveri M., Pignatti P., Vivarelli M., Alonzi T., Fattori E., Garrone S., Barreca A., Martini A. Endocrinology 142, 4818-4826, 2001.
Engelman J A, Berg A H, Lewis R Y, Lisanti M P, and Scherer P E. Tumor necrosis factor alpha-mediated insulin resistance, but not dedifferentiation, is abrogated by MEK1/2 inhibitors in 3T3-L1 adipocytes. Mol Endocrinol 14: 1557-1569, 2000.
Eivindson M., Grønbaek H., Flyvbjerg A., Frystyk J., Zimmermann-Nielsen E., Dahlerup J. F. Growth Horm. IGF Res. 17, 33-40, 2007.
Gao, Z., D. Hwang, F. Bataille, M. Lefevre, D. York, M. J. Quon, and J. Ye. 2002. Serine phosphorylation of insulin receptor substrate 1 by inhibitor κB kinase complex. J. Biol. Chem. 277:48115-48121.
Fernandez-Real, J. M., and Ricart, W. (2003) Endocr. Rev. 24, 278-301
Gu, L., Okada, Y., Clinton, S. K., Gerard, C., Sukhova, G. K., Libby, P., and Rollins, B. J. Mol. Cell 2, 275-281, 1998.
Hotamisligil, G. S., N. S. Shargill, and B. M. Spiegelman. 1993. Adipose expression of tumor necrosis factor-α: direct role in obesity-linked insulin resistance. Science. 259:87-91.
Hotamisligil G S and Spiegelman B M. Tumor necrosis factor alpha: a key component of the obesity-diabetes link. Diabetes 43: 1271-1278, 1994.
Ingermann A R; Yang Y F; Han J; Mikami A; Garza A E; Mohanraj L; Fan L; Idowu M; Ware J L; Kim H S Lee D Y; Oh Y. Identification of a novel cell death receptor mediating IGFBP-3-induced antitumor effects in breast and prostate cancer. J. Biol. Chem, 230(39): 30233-30246, 2010.
Kaplan R C and Frishman W H. Systemic inflammation as a cardiovascular disease risk factor and as a potential target for drug therapy. Heart Dis 3: 326-332, 2001.
Kanety, H., R. Feinstein, M. Z. Papa, R. Hemi, and A. Karasik. 1995. Tumor necrosis factor α-induced phosphorylation of insulin receptor substrate-1 (IRS-1). Possible mechanism for suppression of insulin-stimulated tyrosine phosphorylation of IRS-1. J. Biol. Chem. 270:23780-23784.
Kapur S, Marcotte B, and Marette A. Mechanism of adipose tissue iNOS induction in endotoxemia. Am J Physiol Endocrinol Metab 276: E635-E641, 1999.
Kern S, Eichler H, Stoeve J, Kluter H, Bieback K. Comparative analysis of mesenchymal stem cells from bone marrow, umbilical cord blood, or adipose tissue. Stem Cells 2006; 24:1294-301.
Kershaw, E. E., and Flier, J. S. (2004) J. Clin. Endocrinol. Metab. 89, 2548-2556.
Khanderia U, Pop-Busui R, Eagle K A. Thiazolidinediones in type 2 diabetes: a cardiology perspective. Ann Pharmacother. 2:1466-474, 2009.
Kucerova L, Altanerova V, Matuskova M, Tyciakova S, Altaner C. Adipose tissue-derived human mesenchymal stem cells mediated prodrug cancer gene therapy. J Gene Med. 10:1071-1082, 2008.
Lee, Y. C. et al, WO 2006/085226, US2006-004666, Blockade of Airway Hyperresponsiveness and Inflammation in a Murine Model of Asthma by Insulin-like Growth Factor Binding Protein-3 (IGFBP-3).
Lee Y C, Jogie-Brahim S, Lee D Y; Han J; Harada A; Murphy L J; Oh Y. IGFBP-3 blocks the effects of asthma by negatively regulating NF-B signaling through IGFBP-3R-mediated activation of caspases J. Biol. Chem., 286(20):17898-17909, 2011.
Libby P, Ridker P M, and Maseri A. Inflammation and atherosclerosis. Circulation 105: 1135-1143, 2002.
Linton, M. F., and Fazio, S. (2003) Int. J. Obes. Relat. Metab. Disord. 27, Suppl. 3, 35-40.
Matsuda M, Shimomura I, Sata M, Arita Y, Nishida M, Maeda N, Kumada M, Okamoto Y, Nagaretani H, Nishizawa H, Kishida K, Komuro R, Ouchi N, Kihara S, Nagai R, Funahashi T, and Matsuzawa Y. Role of adiponectin in preventing vascular stenosis. The missing link of adipo-vascular axis. J Biol Chem 277: 37487-37491, 2002.
Ouchi N, Kihara S, Arita Y, Nishida M, Matsuyama A, Okamoto Y, Ishigami M, Kuriyama H, Kishida K, Nishizawa H, Hotta K, Muraguchi M, Ohmoto Y, Yamashita S, Funahashi T, and Matsuzawa Y. Adipocyte-derived plasma protein, adiponectin, suppresses lipid accumulation and class A scavenger receptor expression in human monocyte-derived macrophages. Circulation 103: 1057-1063, 2001.
O'Byrne M B, Inman M D. Airway hyperresponsiveness. Chest 123:411S-416S, 2003.
Ozen M., Cokugras H., Ozen N., Camcioglu Y., Akcakaya N. (2004) Pediatr. Int. 46, 429-435, 2001.
Perreault M and Marette A. Targeted disruption of inducible nitric oxide synthase protects against obesity-linked insulin resistance in muscle. Nat Med 7: 1138-1143, 2001.
Pickup J C, Chusney G D, and Mattock M B. The innate immune response and type 2 diabetes: evidence that leptin is associated with a stress-related (acute-phase) reaction. Clin Endocrinol (Oxf) 52: 107-112, 2000.
Pickup J C, Mattock M B, Chusney G D, and Burt D. NIDDM as a disease of the innate immune system: association of acute-phase reactants and interleukin-6 with metabolic syndrome X. Diabetologia 40: 1286-1292, 1997.
Reaven, G. M. 1988. Role of insulin resistance in human disease. Diabetes. 37:1595-1607.
Ruan H, Hacohen N, Golub T R, Van Parijs L, and Lodish H F. Tumor preadipocyte genes in 3T3-L1 adipocytes: nuclear factor-kappaB activation by TNF-alpha is obligatory. Diabetes 51: 1319-1336, 2002.
Ruan H, Pownall H J, and Lodish H F. Troglitazone antagonizes tumor necrosis factor-alpha-induced reprogramming of adipocyte gene expression by inhibiting the transcriptional regulatory functions of NF-kappaB. J Biol Chem 278: 28181-28192, 2003.
Sartipy, P., and Loskutoff, D. J. (2003) Proc. Natl. Acad. Sci. U.S.A. 100, 7265-7270.
Shimomura, I., Funahashi, T., Takahashi, M., Maeda, K., Kotani, K., Nakamura, T., Yamashita, S., Miura, M., Fukuda, Y., Takemura, K., Tokunaga, K., and Matsuzawa, Y. (1996) Nat. Med. 2, 800-803.
Soukas, A., Cohen, P., Socci, N. D., and Friedman, J. M. (2000) Genes Dev. 14, 963-980.
Weisberg, S. P., McCann, D., Desai, M., Rosenbaum, M., Leibel, R. L., and Ferrante, A. W., Jr. (2003) J. Clin. Investig. 112, 1796-1808.
Strem B M, Hicok K C, Zhu M, et al. Multipotential differentiation of adipose tissue-derived stem cells. Keio J Med 2005; 54:132-41.
Takahashi, K., Mizuarai, S., Araki, H., Mashiko, S., Ishihara, A., Kanatani, A., Itadani, H., and Kotani, H. (2003) J. Biol. Chem. 278, 46654-46660.
Wagner W, Wein F, Seckinger A, et al. Comparative characteristics of mesenchymal stem cells from human bone marrow, adipose tissue, and umbilical cord blood. Exp Hematol. 2005; 33:1402-16.
Weisberg, S. P., Hunter, D., Huber, R., Lemieux, J., Slaymaker, S., Vaddi, K., Charo, I., Leibel, R. L., and Ferrante, A. W. (2006) J. Clin. Investig. 116, 115-124.
Wellen, K. E., and Hotamisligil, G. S. (2003) J. Clin. Investig. 112, 1785-1788.
Wellen, K. E., and Hotamisligil, G. S. (2005) J. Clin. Investig. 115, 1111-1119.
Wong S. C., MacRae V. E., Gracie J. A., McInnes I. B., Galea P., Gardner-Medwin J., Ahmed S. F. (2008) Growth Horm. IGF Res. 18, 369-78
Xu, H., Barnes, G. T., Yang, Q., Tan, G., Yang, D., Chou, C. J., Sole, J., Nichols, A., Ross, J. S., Tartaglia, L. A., and Chen, H. (2003) J. Clin. Investig. 112, 1821-1830.
Yoshimura K, Sato K, Aoi N, Kurita M, Hiroshi T, Harii K. Cell-assisted lipotransfer for cosmetic breast augmentation: supportive use of adipose-derived stem/stromal cells. Aesth. Plast. Surg. 2008; 32:48-55.
Zuk P A, Zhu M, Mizuno H, et al. Multilineage cells from human adipose tissue: implications for cell-based therapies. Tissue Eng. 2001; 7:211-228

What is claimed is:

1. A method for interfering with the activity of nuclear factor-kappaB (NF-κB) in a cell, comprising:
providing to a cell an effective amount of a composition comprising an IGFBP-3 receptor agonist, wherein the IGFBP-3 receptor agonist comprises at least a portion of IGFBP-3, an antibody or a fragment thereof capable of binding at least a portion of an IGFBP-3 receptor, a vector capable of expressing at least a portion of IGFBP-3, or an adenovirus expressing at least a portion of IGFBP-3.

2. (canceled)

3. The method of claim 2, wherein interfering with an activity of NF-κB comprises interfering with a NF-κB signaling pathway.

4-7. (canceled)

8. The method of claim 1, wherein the cell is an adipocyte.

9. The method of claim 1, wherein providing to a cell an effective amount comprises providing from about 0.001 ug to about 1,000 mg/kg subject/day.

10. The method of claim 1, wherein the composition comprises a cell genetically engineered to overexpress at least a portion of IGFBP-3.

11. The method of claim 10, wherein the cell is a mesenchymal stem cell.

12.-32. (canceled)

33. A method for treating a metabolic syndrome, comprising:
administering to a subject having a metabolic syndrome a therapeutically effective amount of a composition comprising an IGFBP-3 receptor agonist, wherein the IGFBP-3 receptor agonist comprises at least a portion of IGFBP-3, an antibody or a fragment thereof capable of binding at least a portion of an IGFBP-3 receptor, a vector capable of expressing at least a portion of IGFBP-3, or an adenovirus expressing at least a portion of IGFBP-3.

34-37. (canceled)

38. The method of claim 33, wherein administering to a subject a therapeutically effective amount of a composition comprises administering from about 0.001 μg to about 1,000 mg/kg subject/day of the composition.

39. The method of claim 33, wherein the metabolic syndrome comprises insulin resistance.

40. The method of claim 39, further comprising decreasing insulin resistance of the subject and increasing uptake of glucose by the subject.

41. The method of claim 40, wherein the increasing uptake of glucose by the subject comprises increasing uptake of glucose by the subject by about at least 100% as compared to a subject having a metabolic syndrome comprising insulin resistance not provided with an effective amount of a composition comprising an IGFBP-3 receptor agonist.

42. The method of claim 33, wherein the metabolic syndrome comprises atherosclerosis.

43. The method of claim 42, further comprising reducing the expression of MCP-1 in the subject.

44. The method of claim 33, wherein the composition comprises a cell genetically engineered to overexpress at least a portion of IGFBP-3.

45. The method of claim 44, wherein the cell is a mesenchymal stem cell.

46-51. (canceled)

52. A method for treating cancer in subject having cancer, comprising:
administering to the subject having cancer a therapeutically effective amount of a composition comprising an IGFBP-3 receptor agonist, wherein the IGFBP-3 receptor agonist comprises at least a portion of IGFBP-3, an antibody or a fragment thereof capable of binding at least a portion of an IGFBP-3 receptor, a vector capable of expressing at least a portion of IGFBP-3, or an adenovirus expressing at least a portion of IGFBP-3.

53. (canceled)

54. The method of claim 52, wherein the vector comprises an adenovirus expressing at least a portion of IGFBP-3.

55. The method of claim 52, wherein the cancer is one of carcinoma, sarcoma, lymphoma and leukemia, germ cell tumor and blastoma.

56. The method of claim 55, wherein the cancer is prostate cancer.
